(19) United States
(12) Patent Application Publication
(10) Pub. No.: US 2025/0272835 A1
(43) Pub. Date: Aug. 28, 2025

(54) PREDICTING TREATMENT EFFICACY BY ANALYZING NON-CANCER CELLS

(71) Applicant: Foundation Medicine, Inc., Cambridge, MA (US)

(72) Inventors: Ericka M. Ebot, Newton, MA (US); Kuei-Ting Chen, Cambridge, MA (US); Mikayla Biggs, Cambridge, MA (US); Douglas I. Lin, Cambridge, MA (US); Julia A. Elvin, Cambridge, MA (US); Garrett Michael Frampton, Somerville, MA (US); James J. Pao, Boston, MA (US)

(73) Assignee: Foundation Medicine, Inc., Cambridge, MA (US)

(21) Appl. No.: 19/064,007

(22) Filed: Feb. 26, 2025

Related U.S. Application Data
(60) Provisional application No. 63/572,777, filed on Apr. 1, 2024.
(60) Provisional application No. 63/559,142, filed on Feb. 28, 2024.

(51) Int. Cl. G06T 7/00; G06T 7/11; G06T 7/13; G06V 10/26; G06V 10/764; G06V 10/77; G06V 20/69; G16B 20/00; G16H 15/00; G16H 20/10; G16H 20/40; G16H 30/40; G16H 50/20

(52) U.S. Cl. CPC G06T 7/0012; G06T 7/11; G06T 7/13; G06V 10/26; G06V 10/764; G06V 10/7715; G06V 20/695; G06V 20/698; G16B 20/00; G16H 15/00; G16H 20/10; G16H 20/40; G16H 30/40; G16H 50/20; G06T 2207/20021; G06T 2207/20081; G06T 2207/20084; G06T 2207/30024; G06T 2207/30096; G06V 2201/03

(57) ABSTRACT

An example method includes identifying an image of a tissue sample obtained from a subject. The tissue sample includes at least one cancer cell and at least one non-cancer cell. The example method further includes identifying the at least one non-cancer cell depicted in the image; generating a metric based on the at least one non-cancer cell depicted in the image; and predicting, based on the metric, whether the at least one cancer cell is susceptible to at least one treatment.

### CROSS-REFERENCE TO RELATED APPLICATIONS

This application claims priority to U.S. Provisional App. No. 63/559,142, which was filed on Feb. 28, 2024, and also to U.S. Provisional App. No. 63/572,777, which was filed on Apr. 1, 2024, each of which is incorporated by reference herein in its entirety.

### BACKGROUND

Clinicians rely on various types of diagnostic tests in order to categorize cancers. For example, clinicians are able to assess cancer types by performing physical exams, medical imaging, histological studies, and other diagnostic analyses. In general, different types of cancer treatments are effective at treating different types of cancers. Accordingly, a clinician may prescribe or administer a cancer treatment to a patient based on the type of cancer cells identified in the patient's body.

Immunotherapies can treat the cancer cells by activating and/or suppressing the immune system of the patient. For instance, particular immune checkpoint inhibitors can be used to induce T cells to destroy PD-L1-expressing cancer cells in the patient's body. A clinician may predict whether a PD-L1-targeting immunotherapy will be effective in treating the cancer cells by determining whether the cancer cells express PD-L1 via immunohistochemistry (IHC). However, PD-L1 expression by the cancer cells may not be determinative of whether a PD-L1-targeting immunotherapy will be effective. For instance, in many cases, PD-L1-targeting immunotherapies are ineffective at treating PD-L1-positive cancer cells.

### BRIEF DESCRIPTION OF THE DRAWINGS

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings, of which:

FIG. 1 illustrates an example environment for determining treatment efficacy based on the presence and amount of non-cancer cells in a tumor microenvironment.

FIG. 2 illustrates an environment with an example of an image analyzer that is also described above with reference to FIG. 1.

FIG. 3 illustrates an example environment for training and utilizing a predictive model to predict whether an anticancer treatment will be effective when administered to a subject.

FIG. 4 illustrates an example report of a cancer of a subject.

FIG. 5 illustrates an example environment for sequencing various nucleic acid molecules.

FIG. 6 illustrates an example process for predicting cancer treatment efficacy based on characteristics of a tumor microenvironment.

FIG. 7 illustrates one or more devices configured to perform various operations described herein.

FIG. 8 summarizes a study population investigated in an Experimental Example described herein.

FIG. 9 illustrates a volcano plot representing HIFs associated with survival in mono-IO cohort and a heatmap of top HIFs observed in the Experimental Example.

FIG. 10 illustrates Kaplan-Meier plots of overall survival for (top) mono-IO and (bottom) chemo-IO treated patients according to HIF status.

FIG. 11 illustrates Kaplan-Meier plots of overall survival for mono-IO treated patients stratified by HIF and TMB status.

FIGS. 12A and 12B illustrate example tissue regions exhibiting strong lymphocyte presence (FIG. 12A) and tumor cells bordered by desmoplastic stroma with interspersed fibroblasts (FIG. 12B).

### DETAILED DESCRIPTION

Various implementations of the present disclosure relate to techniques for predicting the efficacy of a cancer treatment by analyzing features of non-cancer cells. In various cases, an image of a tissue sample that has been stained with a histological stain is obtained. The tissue sample, for instance, is obtained from a tumor of a subject, and includes both cancer cells and non-cancer cells. In various cases, the cancer treatment effectiveness is predicted based, at least in part, on characteristics of the non-cancer cells in the tissue sample. In some examples, one or more metrics quantifying features of the non-cancer cells are calculated based on the image of the tissue sample. For instance, a predictive model is configured to determine whether cancer cells in the subject are susceptible to a particular treatment (e.g., an immunotherapy) based on the metric(s). In some cases, additional features are utilized to predict whether the cancer cells are susceptible to the treatment, such as expression indicators (e.g., results of IHC studies) and/or genomic features of the subject.

Implementations of the present disclosure provide significant improvements to the technical field of cancer diagnostics and therapies. In various cases, metrics associated with the non-cancer cells can greatly enhance the predictive accuracy of whether immunotherapies, and other types of cancer treatments, will be effective when administered to the subject. Accordingly, a clinician relying on various predictions described herein is less likely to prescribe or administer a treatment that will be ineffective. As a result, the subject is less likely to suffer the side effects and financial burdens of treatments that have minimal therapeutic benefit.

Various analyses described herein cannot be performed in the human mind, or by pen and paper. For example, it would not be possible for a human to specifically quantify various characteristics of non-cancer cells in a tissue sample. Moreover, it would not be possible for a human to generate an accurate assessment of a predicted effectiveness of a treatment based on a combination of metrics, expression indicators, and genomic features.

### EXAMPLE DEFINITIONS

As used herein, the term “image,” and its equivalents, may refer to 2D or 3D array of data indicative of an array of pixels or voxels.

As used herein, the term “segmentation,” and its equivalents, can refer to a process of defining an image of into regions. For instance, a segmentation method can be performed by defining an area of a histological image that depicts a cell type (e.g., a non-cancer cell or a cancer cell).

As used herein, the term “segmentation mask,” and its equivalents, can refer to an image that indicates one or more segmented regions of a base image. In some cases, the segmentation mask has the same pixel (or voxel) dimensions as the base image. In some cases, pixels of the segmentation mask have a first value (e.g., 1) if they correspond to pixels of the base image that depict the segmented region(s); and pixels of the segmentation mask have a second value (e.g., 0) if they correspond to pixels of the base image that do not depict the segmented region(s). In some examples, some pixels of the segmentation mask corresponding to the pixels of the segmented region(s) have the same pixel values (e.g., color channels) as the pixels of the base image.

As used herein, the terms “machine learning,” “ML,” “computer learning,” “artificial intelligence,” and their equivalents, may refer to the use of a computing devices to learn patterns in training data. The process of learning these patterns may be referred to as “training.” In particular cases, one or more computing devices may perform machine learning by executing a machine learning model. As used herein, the terms “machine learning model,” “ML model,” and their equivalents, may refer to data encoding instructions that, when executed by at least one computing device, causes the at least one computing device to learn patterns in training data by optimizing one or more metrics, values, or other types of parameters. After training, an ML model, when executed by at least one computing device, causes the at least one computing device to utilize the optimized parameters in order to perform one or more tasks.

As used herein, the terms “convolutional neural network,” “CNN,” and their equivalents, may refer to a computing model defined by multiple convolutional blocks. In various cases, the convolutional blocks of a CNN are arranged in parallel and/or series. In various cases, a CNN includes an input block, one or more convolution blocks, and an output block.

As used herein, the terms “blocks,” “layers,” and the like can refer to devices, systems, and/or software instances (e.g., Application Programming Interfaces (APIs), Virtual Machine (VM) instances, or the like) that generates an output by apply an operation to an input. A “convolutional block,” for example, can refer to a block that applies a convolution operation to an input (e.g., an image). When a first block is in series with a second block, the first block may accept an input, generate an output by applying an operation to the input, and provide the output to the second block, wherein the second block accepts the output of the first block as its own input. When a first block is in parallel with a second block, the first block and the second block may each accept the same input, and may generate respective outputs that can be provided to a third block. In some examples, a block may be composed of multiple blocks that are connected to each other in series and/or in parallel. In various implementations, one block may include multiple layers.

In some cases, a block can be composed of multiple neurons. As used herein, the term “neuron,” or the like, can refer to a device, system, and/or software instance (e.g., VM instance) in a block that applies a kernel to a portion of an input to the block.

As used herein, the term “kernel,” and its equivalents, can refer to a function, such as applying a filter, performed by a neuron on a portion of an input to a block.

As used herein, the term “pixel,” and its equivalents, can refer to a value that corresponds to an area or volume of an image. In a grayscale image, the value can correspond to a grayscale value of an area of the grayscale image. In a color image, the value can correspond to a color value of an area of the color image. In a binary image, the value can correspond to one of two levels (e.g., a 1 or a 0). The area or volume of the pixel may be significantly smaller than the area or volume of the image containing the pixel. In examples of a line defined in an image, a point on the line can be represented by one or more pixels. A “voxel” is an example of a pixel spatially defined in three dimensions.

As used herein, the term “receptive field,” and its equivalents, can refer to a group of pixels input into a neuron of a NN.

As used herein, the term “dilation rate,” and its equivalents, can refer to gaps between pixels in the receptive field of a neuron that are ignored by the neuron. For example, the gaps may not be convolved or cross-correlated with the kernel of the neuron.

As used herein, the terms “deoxyribonucleic acid,” “DNA,” “DNA molecule,” and their equivalents, may refer to a polymer of nucleotides (also referred to as “nucleobases”) containing deoxyribose. The nucleotides in DNA include cytosine (C), guanine (G), adenine (A), and thymine (T). Each DNA nucleotide includes a deoxyribose and a phosphate group. An example single-stranded DNA (ssDNA) molecule includes a chain of covalently bonded DNA nucleotides. In the example ssDNA molecule, the phosphate group of the mth nucleotide is covalently bonded to the deoxyribose of the (m−1)th nucleotide, wherein m is a positive integer greater than 2 and less than or equal to the number of DNA nucleotides in the chain. In various examples, DNA is double-stranded and includes two ssDNA molecules that are complementary to one another and coiled around each other in a double helix form. The nucleotides of one ssDNA molecule are hydrogen bonded to the nucleotides of the other ssDNA molecule. In particular, the pyrimidines (A and T) hydrogen bond to each other, and the purines (C and G) hydrogen bond to each other.

As used herein, the terms “ribonucleic acid,” “RNA,” “RNA molecule,” and their equivalents, may refer to a polymer of nucleotides containing ribose. The nucleotides in RNA include cytosine (C), guanine (G), adenine (A), and uracil (U). Each RNA nucleotide includes a ribose and a phosphate group. In an example RNA molecule, the phosphate group of the nth nucleotide is covalently bonded to the ribose of the (n−1)th nucleotide, wherein n is a positive integer greater than 2 and less than or equal to the number of RNA nucleotides in the chain. Messenger RNA (mRNA) is a type of RNA molecule that is synthesized (or “transcribed”) by RNA polymerase (an enzyme) to be complementary to a gene encoded in a DNA sequence, and is also used by a ribosome to synthesize a polypeptide or protein. An mRNA is therefore an example of a “coding RNA.” In various cases, intron sequences are removed from an mRNA via a process known as “RNA splicing.” MicroRNA (“miRNA”) are single-stranded RNA molecules that perform post-transcriptional gene expression regulation. For instance, a miRNA may bind to a complementary mRNA molecule, thereby cleaving, destabilizing, or otherwise preventing the mRNA molecule from being translated into a polypeptide or protein by a ribosome. In various examples, a miRNA has a length in a range of 21 to 23 RNA nucleotides. As used herein, the terms “non-coding RNA” may refer to a type of RNA that is not translated into a protein. Examples of non-coding RNA include miRNA, transfer RNA (tRNA), and ribosomal RNA (rRNA). The term “functional RNA,” and its equivalents, may refer to any RNA molecule that impacts a biological process. For instance, functional RNA may include mRNA, miRNA, tRNA, rRNA, and the like.

As used herein, the term “base,” and its equivalents, may refer to a monomer of a polymer. For example, a base of DNA or RNA is a nucleotide.

As used herein, the term “base pair,” and its equivalents, may refer to a pair of complementary DNA nucleotides, which are hydrogen-bonded to one another in a double-stranded DNA molecule. For example, a base pair includes a first base in a first ssDNA and a second base in a second ssDNA, wherein the first and second bases are complementary and hydrogen-bonded to one another.

As used herein, the terms “nucleotide,” “nucleobase,” “nucleic acid,” “nucleic acid molecule,” and their equivalents, may refer to an organic molecule that includes a nitrogenous base, a sugar, and a phosphate group. In various cases, a nucleotide is a monomer of DNA or RNA. A nucleotide, for instance, is a chemical structure.

As used herein, the terms “3′ end,” “3-prime end,” and their equivalents, may refer to a terminus of a single-stranded nucleotide polymer that includes a base whose third carbon in its deoxyribose or ribose is bound to a hydroxyl group while being unbound to another base.

As used herein, the terms “5′ end,” “5-prime end,” and their equivalents, may refer to a terminus of a single-stranded nucleotide polymer that includes a base whose fifth carbon in its deoxyribose or ribose ring is unbound to another base. In some cases, the fifth carbon is bound to a phosphate group.

As used herein, the “length” of a polymer refers to a number of covalently bonded monomers that are included in the polymer. For instance, the length of a DNA molecule may be the number of covalently bonded nucleotides in at least one strand of the DNA molecule and/or the number of base pairs in the DNA molecule. In various examples, the length of an RNA molecule may be the number of covalently bonded nucleotides in the RNA molecule.

As used herein, the term “gene,” and its equivalents, refers to a sequence of DNA nucleotides that is transcribed into a functional RNA. The functional RNA, for instance, is RNA that is translated into a polypeptide or protein (e.g., mRNA) or that has some other biological function (e.g., miRNA, tRNA, etc.). A gene is “expressed” when it is used as a template to generate a functional RNA. A subject, for instance, has numerous genes contained in the subject's genome. A gene may include both introns and exons. As used herein, the term “intron,” and its equivalents, may refer to a subset of DNA nucleotides in a gene that is not used to code for any functional RNA that is expressed by the organism. As used herein, the term “exon,” and its equivalents, may refer to a subset of DNA nucleotides in a gene that is used to code for a functional RNA. For instance, an exon may encode a polypeptide or protein that is expressed by the organism. In various examples, a gene can be represented in data (e.g., as data representative of the sequence of DNA nucleotides in the gene) or as a chemical structure (e.g., as the sequence of DNA nucleotides itself).

As used herein, the term “genome,” and its equivalents, refers to the aggregate of genes of a subject. In various cases, a genome represents the sequences of several linear DNA molecules that are present in a subject's chromosomes. A “reference genome” refers to an aggregation of genes of one or more reference subjects. In various cases, a genome is represented in data.

As used herein, the terms “pangenome,” “pan-genome,” “supragenome,” and their equivalents, refers to an aggregate set of genes from multiple subgroups (e.g., strains) within a population (e.g., a clade) of subjects. A pangenome, for example, indicates genes that are present in all subjects within the population, as well as genes that are present in some of the subjects of the population. A pangenome is represented in data, for instance.

As used herein, the term “transcriptome,” and its equivalents, refers to the aggregate of RNA sequences of a subject. In some cases, a transcriptome is limited to mRNA sequences. In various examples, a transcriptome is represented in data.

As used herein, the term “genomic DNA,” “gDNA,” “chromosomal DNA,” and their equivalents, may refer to DNA molecules that are obtained from a chromosome and/or nucleus of a cell.

As used herein, the terms “DNA fragment,” “fragment,” and their equivalents, may refer to DNA molecules that are excised and/or broken off from a larger DNA molecule.

As used herein, the terms “cell-free DNA,” “cfDNA,” and their equivalents, may refer to DNA fragments that are non-encapsulated and obtained outside of cells within a sample (e.g., a liquid biopsy sample).

As used herein, the terms “circulating tumor DNA,” “ctDNA,” and their equivalents, may refer to a cfDNA molecule that originates from a cancer cell.

As used herein, the terms “end motif,” “terminal sequences,” and their equivalents, may refer to a sequence of nucleotides extending from a 3′ or 5′ end of a DNA or RNA molecule. In various cases, the end motif is shorter than a length of the DNA or RNA molecule. For example, the end motif may have a length in a range of 5 to 30 bases or base pairs, a range of 3 to 30 bases or base pairs, or a range of 1 to 30 base pairs.

As used herein, the term “promoter,” and its equivalents, may refer to a portion of a DNA molecule that binds one or more proteins in order to initiate transcription of a gene. For example, the promotor is located “upstream” of the gene. For example, the promotor is located between 5′ end of the DNA molecule and the gene. A promotor may include one or more binding sites for RNA polymerase, and/or one or more transcription factor binding sites. In some examples, a promotor includes one or more CpG islands. A promoter, for instance, includes a transcription start site.

As used herein, the terms “CpG island,” “CGI,” “CpG site,” and their equivalents, may refer to a continuous portion of a DNA molecule whose sequence includes greater than a threshold amount (e.g., greater than 50%) of G-C base pairs.

As used herein, the term “enhancer,” and its equivalents, may refer to a portion of a DNA molecule that binds one or more proteins in order to increase the chance that a gene will be transcribed. For instance, an enhancer includes one or more transcription factor binding sites. In various cases, an enhancer includes one or more CpG islands.

As used herein, the term “cancer,” and its equivalents, may refer to a condition of a subject in which particular cells (referred to as “cancer cells”) divide uncontrollably in the subject's body. In some cases, a cancer is characterized by a location or tissue type from which the cancer cells originated. In some examples, a cancer is characterized by a location or tissue type in which the cancer cells are located.

As used herein, the terms “tumor,” “neoplasm,” and their equivalents, may refer to a mass of tissue including cancer cells.

As used herein, the terms “tissue of origin,” “tissue origin,” and their equivalents, refers to a differentiated type of tissue from which cancer cells in the body of a subject began dividing uncontrollably in the subject's body.

As used herein, the terms “liquid biopsy,” “fluid biopsy,” and their equivalents, may refer to a process of obtaining a fluid sample from a subject's body. The sample, for instance, can be referred to as a “liquid biopsy sample.” Examples of fluids that are sampled from the body include blood, plasma, cerebrospinal fluid, sputum, stool, urine, lymphatic fluid, and saliva.

As used herein, the term “tissue biopsy,” and its equivalents, may refer to a process of obtaining a sample of cells from a subject's body. A tissue biopsy, in various cases, is performed by cutting a mass of cells from the subject's body. For instance, a tissue biopsy is a procedure performed by a surgeon, interventional radiologist, interventional cardiologist, or other specialized clinician. The term “tissue” or “tissue biopsy sample” can be used to refer to the sample of cells obtained using a tissue biopsy.

As used herein, the term “subject,” and its equivalents, may refer to a human or non-human animal. A subject that is receiving care from at least one care provider may be referred to as a “patient.”

As used herein, the term “variant,” and its equivalents, may refer to a difference between a subject genetic sequence and a reference sequence. For instance, a variant may correspond to a difference between one or more nucleotides in a genome of a subject and one or more corresponding nucleotides in at least one reference genome or pangenome. A variant may be characterized by its identity (e.g., what nucleotides are different), its position (e.g., where are the nucleotides located in the genome, what chromosome contains the nucleotides, what gene contains the nucleotides, etc.), its length (e.g., how many nucleotides are different from the reference sequence), its type (e.g., substitution, insertion, deletion, copy number alternation, rearrangement of fusion, etc.), and other features that indicates its significance and/or relevance. In some cases, a variant represents any apparent alteration in a sequence that has been read from a nucleic acid molecule with respect to the reference sequence, such as reads cleaved by restriction enzymes (RE). In various examples, a variant can be represented in data (e.g., by data characterizing the variant) or as a chemical structure (e.g., the nucleotides themselves). As used herein, the term “mutation,” and its equivalents, may refer to a change in a gene.

As used herein, the term “substitution,” and its equivalents, can refer to a nucleotide in a subject sequence that is different than an equivalent nucleotide (e.g., a nucleotide at the same position) in a reference sequence.

As used herein, the term “insertion,” and its equivalents, can refer to a nucleotide in a subject sequence that is added with respect to a reference sequence.

As used herein, the term “deletion,” and its equivalents, can refer to the removal of a nucleotide from a nucleotide sequence.

As used herein, the terms “copy number alternation,” “CNA,” “copy number variation,” “CNV,” and their equivalents, can refer to a portion of a reference sequence that is repeated.

As used herein, the terms “rearrangement of fusion,” “fusion rearrangement,” “translocation,” and their equivalents, can refer to a change in the relative position of one or more portions of a reference sequence, thereby generating a gene that was not present in the reference sequence.

As used herein, the term “sequencing,” and its equivalents, may refer to a process of identifying the order and identity of monomers in a polymer chain, such as the order and identity of nucleotides in a DNA or RNA molecule. The terms “whole genome sequencing,” “WGS,” and their equivalents, may refer to the process of sequencing an entire genome of a subject, including the introns and exons of the genes of the subject. The term “whole exome sequencing,” and its equivalents, may refer to the process of sequencing all exomes of a subject. The term “targeted sequencing,” and its equivalents, may refer to the process of sequencing a portion of the genome of a subject, such as sequencing a single gene of the subject. Various techniques can be utilized to sequence a DNA or RNA molecule, such as massively parallel sequencing (MPS), nanopore sequencing, direct sequencing, Sanger sequencing, or next-generation sequencing. In various cases, sequencing is performed on physical molecules (e.g., RNA or DNA) and is used to generate data.

As used herein, the terms “massive parallel sequencing,” “massively parallel sequencing,” “MPS,” and their equivalents, may refer to a technique for simultaneously performing multiple reactions that can be used to identify the order and identity of monomers in multiple polymer chains. In particular cases, massive parallel sequencing can be performed using sequencing-by-synthesis on clonally amplified DNA molecules that are located in spatially separated regions, which are individually monitored by sensors.

As used herein, the term “nanopore sequencing,” and its equivalents, may refer to a technique for identifying the order and identity of monomers in a polymer chain by transporting the polymer chain from a first space to a second space, wherein the first space and the second space are separated by a substrate, by directing the polymer chain through a small hole (known as a “nanopore”) embedded in the substrate, and monitoring a relative electrical signal (e.g., a voltage or current) between the first space and the second space.

As used herein, the term “sensor,” and its equivalents, may refer to a physical device or other apparatus that is configured to detect one or more detection signals.

As used herein, the term “detection signal,” and its equivalents, may refer to a physical signal that can be identified, characterized, or otherwise perceived by a sensor.

As used herein, the term “sequence read data,” and its equivalents, may refer to data that is indicative of an order and identity of monomers in a polymer, such as the order and identity of nucleotides in a DNA or RNA sequence. In various implementations, sequence read data is generated via a sequencing operation.

As used herein, the term “ligating,” and its equivalents, may refer to a process of joining two molecules together, for example, with a chemical bond.

As used herein, the term “adapter,” and its equivalents, may refer to an oligonucleotide that can be ligated to a target nucleic acid molecule. In various cases, an adapter prepares the target nucleic acid molecule for sequencing.

As used herein, the term “bait molecule,” and its equivalents, may refer to a nucleic acid molecule having a region that is complementary to a region of a target molecule (e.g., cfDNA). A bait molecule includes, for instance, a nucleic acid molecule that can hybridize to (i.e., is complementary to) a target molecule can be used to capture the target molecule. In some instances, the bait molecule is a capture oligonucleotide (or capture probe). In some instances, the bait molecule is suitable for solution phase hybridization to the target molecule. In some instances, the bait molecule is suitable for solid phase hybridization to the target molecule. In some instances, the bait molecule is suitable for both solution-phase and solid-phase hybridization to the target molecule. The design and construction of bait molecules is described in more detail in, e.g., International Patent Application Publication No. WO 2020/236941.

As used herein, the term “amplifying,” and its equivalents, may refer to a process of generating copies of a target molecule, such as a nucleic acid molecule.

As used herein, the term “hybridization,” and its equivalents, may refer to a process by which to complementary single-stranded nucleic acid molecules bind to one another, thereby forming a double-stranded nucleic acid molecule. In certain examples, the double-stranded nature of the nucleic acid molecule is maintained under stringent hybridization conditions. Exemplary stringent hybridization conditions include an overnight incubation at 42° C. in a solution including 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at 50° C.

As used herein, the term “complementary,” and its equivalents, may refer to a state of two single-stranded nucleic acid molecules with respective sequences that cause the nucleic acid molecules to spontaneously hybridize to one another. One nucleic acid molecule, for instance, may have a sequence that causes each nucleic acid to hydrogen bond to a respective nucleic acid in the other nucleic acid molecule.

As used herein, the terms “therapy,” “treatment,” and their equivalents, may refer to a composition or process that can be used to remediate a health problem. Cancer therapies (also referred to as “anti-cancer therapies”), for instance, include surgery, radiotherapy, chemotherapy, immunotherapy, cell-based therapies, and the like. Examples of cancer therapies include abemaciclib (Verzenio), abiraterone acetate (Zytiga), acalabrutinib (Calquence), ado-trastuzumab emtansine (Kadcyla), afatinib dimaleate (Gilotrif), aldesleukin (Proleukin), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), copanlisib hydrochloride (Aliqopa), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), iobenguane I131 (Azedra), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), Lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximabcmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), panobinostat (Farydak), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecanhziy (Trodelvy), seliciclib, selinexor (Xpovio), selpercatinib (Retevmo), selumetinib sulfate (Koselugo), siltuximab (Sylvant), sipuleucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), and combinations thereof. Examples of cancer therapies also include targeted antibody-based therapies (antibody-drug conjugates, antibody-radioisotope conjugates, and targeted immune cell therapies (e.g., immune effector cells genetically modified to express a chimeric antigen receptor (CAR).

As used herein, the term “treatment-responsive,” and its equivalents, may refer to a type of cancer cell that can be substantially killed using a predetermined type of therapy. For example, cancer cells of a subject may be responsive to a particular treatment if, after the subject is administered the treatment, the cancer cells are diminished by a particular progression level (e.g., radiographic progression level, marker-based progression level, such as prostate-specific antigen (PSA) progression, etc.). Accordingly, the responsiveness of the cells to the type of therapy may indicate the effectiveness of that therapy. Cancer cells are “treatment-responsive” to a treatment if they are susceptible to the treatment.

As used herein, the term “treatment-resistant,” and its equivalents, may refer to a type of cancer that cannot be substantially killed using a predetermined type of therapy.

As used herein, the term “metastasis profile,” and its equivalents, may refer to a propensity of a type of cancer to metastasize into one or more differentiated tumor types besides the cancer's tissue origin. In some implementations, the metastasis profile can further indicate the type of tissue in which the cancer can or is likely to metastasize.

As used herein, the term “clinical trial,” and its equivalents, may refer to a research study used to evaluate a hypothesis based on participation by one or more subjects. In various examples, a clinical trial can be used to assess the efficacy and/or safety of a proposed therapy. A clinical trial may be performed in furtherance of approval of a treatment by a regulatory authority (e.g., the United States Food & Drug Administration (FDA)).

### DESCRIPTION OF EXAMPLE IMPLEMENTATIONS

Various implementations of the present disclosure will now be described with reference to the accompanying Figures.

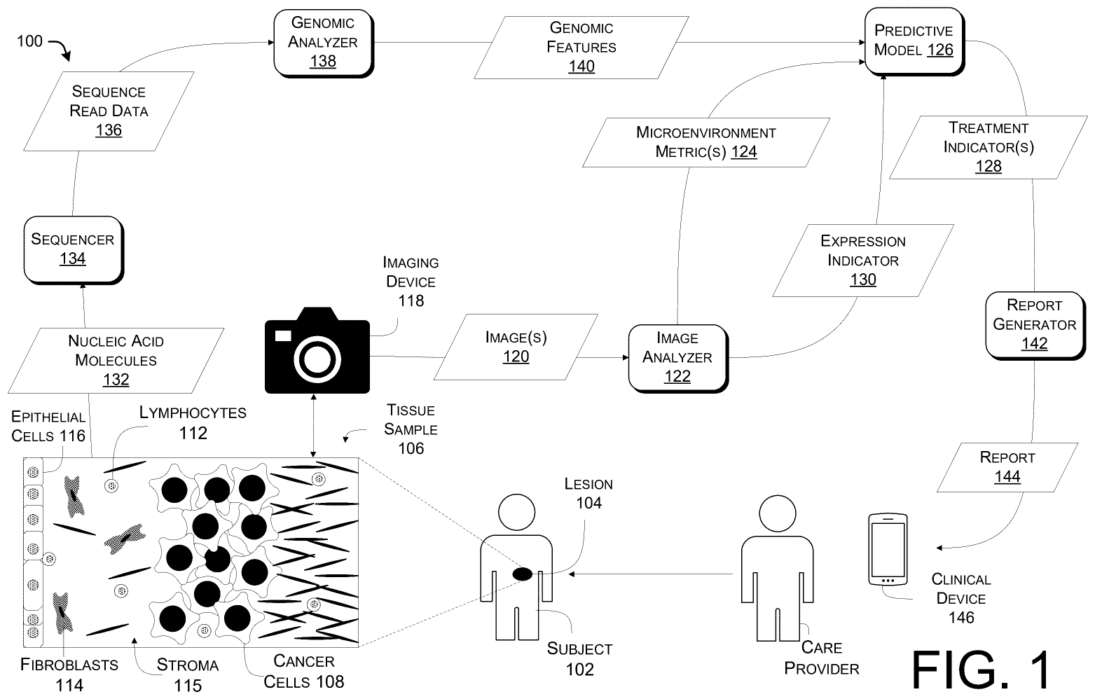

FIG. 1 illustrates an example environment 100 for determining treatment efficacy based on the presence and amount of non-cancer cells in a tumor microenvironment.

A subject 102, for instance, may present to a clinical environment with a lesion 104. In various cases, the lesion 104 may be a tumor that includes cancer cells. According to various examples, the subject 102 has one or more types of cancer, such as adrenal cancer, bladder cancer, blood cancer, bone cancer, brain cancer, breast cancer, carcinoma, cervical cancer, colon cancer, colorectal cancer, corpus uterine cancer, ear, nose and throat (ENT) cancer, endometrial cancer, esophageal cancer, gastrointestinal cancer, head and neck cancer, Hodgkin's disease, intestinal cancer, kidney cancer, larynx cancer, leukemia, liver cancer, lymph node cancer, lymphoma, lung cancer, melanoma, mesothelioma, myeloma, nasopharynx cancer, a neuroblastoma, non-Hodgkin's lymphoma, oral cancer, ovarian cancer, pancreatic cancer, penile cancer, pharynx cancer, prostate cancer, rectal cancer, sarcoma, seminoma, skin cancer, stomach cancer, a teratoma, testicular cancer, thyroid cancer, uterine cancer, vaginal cancer, a vascular tumor, or combinations or metastases thereof.

In some embodiments, the subject 102 has a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

In some embodiments, the subject 102 has acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR/MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), a non-small cell lung cancer KRAS (+/−G12C), a non-small cell lung cancer TMB-H, a non-small cell lung cancer MET exon 14 skipping, a non-small cell lung cancer ERBB2 inframe indel, a non-small cell lung cancer EGFR exon 20 indel, a neurotrophic tyrosine receptor kinase (NTRK)-positive cancer, ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

In various cases, a care provider 105 is responsible for diagnosing and/or treating the subject 102. According to some implementations, the lesion 104 may be initially identified using a noninvasive technique. For example, the lesion 104 may be visualized using an imaging modality, such as ultrasound, x-ray, computed tomography (CT), magnetic resonance imaging (MRI), positron emission tomography (PET), single photon emission CT (SPECT), or any combination thereof. Using the noninvasive technique, the care provider 105 may identify the presence of the lesion 104, but may be unable to determine whether the lesion 104 is a cancerous tumor using noninvasive diagnostic methodologies. In some cases in which the lesion 104 is a tumor, the care provider 105 may be unable to identify whether the tumor is metastatic or benign, or may be unable to otherwise categorize the tumor. Certain types of cancer therapies, for instance, are ineffective for treating particular types of cancer. In various examples, the care provider 105 may be unable to determine an effective therapy to target the lesion 104 without further analysis.

In various implementations, the care provider 105 performs further analysis on the lesion 104 by initiating a tissue biopsy on the subject 102. Tissue biopsies, for example, can include endoscopic biopsies, core needle biopsies, fine-needle aspiration, shave biopsies, punch biopsies, incisional biopsies, excisional biopsies, bone marrow biopsies, surgical biopsies, and the like. For instance, the care provider 105 surgically removes a tissue sample 106 from the lesion 104.

The tissue sample 106 includes various types of cells and structures that are relevant to classifying the lesion 104 and for predicting which treatments would be effective in treating the lesion 104. In various cases, the tissue sample 106 includes multiple cancer cells 108. The cancer cells 108, for example, are cells that divide uncontrollably within the lesion 104. In various cases, they form the lesion 104 as a solid tumor. The cancer cells 108 may have various distinct histological features, such as large nuclei, multiple nuclei, small cytoplasm, irregular outlines, and the like. In various cases, the cancer cells 108 may originate from one or more types of tissues.

Further, the tissue sample 106 includes non-cancer cells. The terms “non-cancer cell,” “non-tumor cell,” and their equivalents, may refer to a cell that is not cancerous. For example, the tissue sample 106 includes lymphocytes 112. The lymphocytes 112 are immune cells. In some cases, the lymphocytes 112 include B cells, T cells, innate lymphoid cells (ILCs), or any combination thereof. The lymphocytes 112 exhibit specific visual characteristics, such as a dense nucleus with minimal cytoplasm. In addition, the lymphocytes 112 may express one or more ligands, such as CD3, CD 4, CD6, CD16, CD19, CD20, TCRαβ, TCRγδ, or any combination thereof. Functionally, the lymphocytes 112 are part of the immune system and enable immune responses. Some lymphocytes 112, for instance, are cytotoxic and can kill cells when particular conditions are satisfied.

In various cases, the tissue sample 106 includes fibroblasts 114. The fibroblasts 114, for instance, may be located in stroma 115 within the tissue sample 106. Fibroblasts 114 produce extracellular matrix and collagen within an organism. In various cases, the fibroblasts 114 produce the stroma 115. Fibroblasts 114, in various cases, have a key role in immune response. The stroma 115 includes structural portions of tissue, rather than functional cells (e.g., the parenchyma). Visually, fibroblasts 114 may have elongated shapes with a branched cytoplasm and a speckled nucleus.

According to some examples, the tissue sample 106 includes epithelial cells 116. The epithelial cells 116 include cells that line the outer and/or inner surfaces of organs, blood vessels, and other cavities within the subject 102. In general, the epithelial cells 116 may present within the tissue sample 106 in one or more closely packed layers referred to as “epithelium.” Epithelial cells 116, in some cases, express immune mediators and other ligands relevant to immune response. In various cases, epithelial cells 116 that make up blood vessel walls are indicative of vascular perfusion to the lesion 104 and/or tissue sample 106.

Although not specifically illustrated in FIG. 1, the tissue sample 106 may include additional non-cancer cells. For instance, non-cancer cells can include adrenal cells, adipocytes, barrier cells, blood cells, bone marrow cells, erythrocytes (also referred to as “red blood cells”), fibroblasts, germ cells, endocrine cells, epidermal cells, ependymal cells, extracellular matrix cells, lens cells, hepatocytes, platelets, neurons, glia, muscle cells, thyroid cells, immune cells, islet cells, interstitial cells, pituitary cells, oral cells, secretory cells, sensory transducer cells, or any combination thereof.

Traditionally, the care provider 105 may predict whether a particular treatment, such as an immunotherapy, would be effective in treating the cancer cells 108 by performing histochemistry and/or IHC. In general, the tissue sample 106 would be preserved using one or more fixatives. In various examples, the tissue sample 106 is immersed into a solution that includes the fixative(s). Examples of the fixative(s) include, for instance, formaldehyde, formalin, hepes-glutamic acid buffer-mediated organic solvent protection effect (HOPE) fixative, a picrate, Zenker's fixative, B-5, potassium dichromate, chromic acid, potassium permanganate, glutaraldehyde, an alcohol, or any combination thereof. The process of fixation prevents degradation of the tissue sample.

In some cases, the tissue sample 106 is dehydrated. In some cases, a chemical (e.g., ethanol) is used to remove water from the tissue sample 106 after fixation. In some cases, additional additives (e.g., resins and/or waxes) are introduced to the tissue sample 106 to solidify the tissue sample 106. In some cases, the tissue sample 106 is cut into sections for further examination.

In various cases, the tissue sample 106 is subjected to one or more stains. Histological stains include chemical dyes that enhance the visibility of portions of the tissue sample 106 using light microscopy. Examples of histological stains include, for instance, alcian blue, aldehyde fuchsin, eosin, giemsa, hematoxylin, van Gieson stain, toluidine blue, reticulin, Nissl stain, (e.g., cresyl echt violet acetate), orcein, sudan black B, trichrome stains (e.g., Masson's trichrome, Mallory's trichrome, Azan trichrome, Cason's trichrome, etc.), periodic acid Schiff, Weigert's resorcin fuchsin, Wright stain, Wright giemsa stain, and the like. When used together, hematoxylin and eosin are referred to as H&E. For example, histological stains enable the care provider 105 to visually identify relevant structures in the tissue sample 106 by analyzing their morphological features.

In some examples, the tissue sample 106 is stained with one or more immunostains. The terms “immunostain,” “immune-stain,” “immunohistostain,” “IHC stain,” and their equivalents, refer to a dye attached to a targeting structure (e.g., antibody or aptamer) that specifically binds a ligand of interest. In various cases, the dye includes a chromogen and/or a fluorescent dye that emits a visible color or light signal. In various cases, the targeting structure of the immunostain is one or more molecules that include a binding domain that specifically binds a ligand that is expressed by one or more types of cancer cells. For instance, the targeting structure may specifically bind a protein that is differentially expressed by a cancer cell.

In various cases, the tissue sample 106 can be stained with different types of stains. For example, a section of the tissue sample 106 may be stained with H&E, washed, and then subsequently stained with an immunostain. In some cases, a first section of the tissue sample 106 can be stained with a first stain (e.g., H&E) and a second section of the tissue sample 106 can be stained with a second stain (e.g., an immunostain). Thus, different types of studies can be performed on the same sample.

In various implementations, histological and immunohistological studies may be performed on the tissue sample 106 in order to assess whether the cancer cells 108 are present in the tissue sample 106. For instance, the care provider 105 may manually identify the presence of the cancer cells 108 in the tissue sample 106 by reviewing an H&E section of the tissue sample 106. Further, the care provider 105 may determine that the cancer cells 108 express a particular antigen of interest by determining that the cancer cells 108 fluoresce after being stained with an immunostain having a fluorescent dye bound to an antibody that specifically binds the antigen.

In some cases, the care provider 105 may predict that the cancer cells 108 are susceptible to a particular treatment based on reviewing histological and immunohistological characteristics of the cancer cells 108. For instance, if an immunohistological assay indicates that the cancer cells 108 express a particular antigen that is a target of a particular immunotherapy, then the care provider 105 may infer that the cancer cells 108 are susceptible to the immunotherapy. In some cases, if the immunohistological assay indicates that the cancer cells 108 do not express the particular antigen, then the care provider 105 may infer that the cancer cells 108 are not susceptible to the immunotherapy. If the cancer cells 108 are susceptible to the immunotherapy, then the care provider 105 may prescribe and/or administer the immunotherapy to the subject 102. If the cancer cells 108 are not predicted to be susceptible to the immunotherapy, then the care provider 105 may prescribe, recommend, or administer a different type of therapy (e.g., radiation therapy, chemotherapy, or the like).

However, immunohistological assays are not necessarily definitive evidence of whether a cancer treatment will be effective. Various characteristics of the microenvironment within the tissue sample 106 (e.g., the tumor microenvironment, if the lesion 104 is a tumor) impact whether a particular treatment will successfully reduce or eliminate the presence of the cancer cells 108 in the body of the subject 102.

For instance, an example immunotherapy may include an antibody with a binding domain that specifically binds an antigen expressed by the cancer cells 108. The immunotherapy may be administered to the subject 102 intravenously. However, if the cancer cells 108 are not located near blood vessels through which the immunotherapy travels, then the immunotherapy may be unable to bind to the cancer cells 108 due to the vascular physiology of the subject 102 and/or the lesion 104. In these cases, an immunohistological study may indicate that the cancer cells 108 express an antigen that is targeted by the immunotherapy, but the immunotherapy may nevertheless be ineffective at treating the cancer cells 108 in the lesion 104.

Furthermore, in various cases, the effectiveness of the immunotherapy is dependent on the actions of immune cells. For example, once the immunotherapy binds to the cancer cells 108, the immunotherapy may cause the lymphocytes 112 to kill the cancer cells 108 based on the presence of the immunotherapy. However, if the lymphocytes 112 are unable to, or rarely, physically encounter the cancer cells 108 in the lesion 104, then the cancer cells 108 would not be effectively destroyed by the lymphocytes 112. Thus, even if the immunohistological study indicates that the cancer cells 108 express an antigen that is targeted by the immunotherapy, the immunotherapy may not treat the cancer cells 108 in the lesion 104 if the lesion 104 lacks a sufficient amount of the lymphocytes 112.

The subject 102 may suffer significant problems due to receiving an ineffective treatment for the cancer cells 108 in the lesion. Various cancer treatments are associated with significant and dangerous side-effects, such as fatigue, nausea, hair loss, infections, anemia, bruising, bleeding, injection-site pain, rashes, blisters, chills, constipation, coughing, decreased appetite, and fever. Moreover, various cancer treatments can be associated with a significant financial cost. In some cases, the risk of the side-effects, and the cost of the treatments, are outweighed by their efficacy at treating the cancer cells 108. However, if a treatment is ineffective at treating the cancer cells 108, then administering the treatment may cause significant and unnecessary harm to the subject 102. Thus, there is a need to enhance the predictive accuracy of cancer treatment efficacy.

In various implementations of the present disclosure, the predictive accuracy that a given treatment will successfully treat the cancer cells 108 can be increased by analyzing characteristics of non-cancer cells in the tissue sample 106. Using various techniques described herein, the care provider 105 may more confidently prescribe, recommend, or administer a treatment that will treat the cancer cells 108 of the subject 102. In addition, the care provider 105 may more confidently refrain from prescribing, recommending, or administering a treatment that will not treat the cancer cells 108. Accordingly, unnecessary physical harm (due to side effects of unnecessary treatments) and unnecessary financial harm (due to the costs of unnecessary treatments) to the subject 102 can be prevented.

In various implementations of the present disclosure, an imaging device 118 captures at least one image 120 of the tissue sample 106. In various cases, the tissue sample 106 is transported to a location that is remote from the subject 102 for further processing. For example, the tissue sample 106 is removed from the subject 102 in a clinical environment (e.g., a hospital) and is then transported to a remote laboratory including the imaging device 118 for further testing and analysis. The imaging device 118, for instance, is a camera including various photosensors configured to detect light in a field-of-view (FOV) that includes at least one portion of the tissue sample 106. According to some cases, the imaging device 118 includes one or more lenses that enable the imaging device 118 to capture the image(s) 120 from a magnified FOV. In some cases, the imaging device 118 includes one or more light sources configured to illuminate the tissue sample 106 during image acquisition.

The image(s) 120 depict the tissue sample 106 stained with one or more histological and/or immunohistological stains. For example, the image(s) 120 may depict the tissue sample 106 stained with H&E or some other histological stain. In some cases, the image(s) 120 further depict the tissue sample 106 stained with an immunostain targeting a particular ligand-of-interest.

According to some examples, the image(s) 120 include one or more 2D images and/or 3D images. In some cases, the image(s) 120 include multiple 2D images depicting multiple slices of the tissue sample 106. In some cases, the multiple 2D images are stacked into a 3D image for further processing.

An image analyzer 122 is configured to analyze the image(s) 120. In various cases, the image analyzer 122 is configured to evaluate features of a microenvironment of a tumor from which the tissue sample 106 is obtained. In various cases, the image analyzer 122 is configured to generate at least one microenvironment metric 124 by analyzing the image(s) 120. The microenvironment metric(s) 124, for instance, are representative of non-cancer cells in the tissue sample 106.

In some examples, the image analyzer 122 identifies the non-cancer cells in the tissue sample 106. For example, the image analyzer 122 identifies at least one of the lymphocytes 112, the fibroblasts 114, or the epithelial cells 116 depicted in the image(s) 120. In some cases, the image analyzer 122 performs image segmentation on the image(s) 120. The term “image segmentation,” for instance, can refer to a process of partitioning the pixels or voxels of an image into multiple regions. In some cases, one or more of the regions can be extracted or otherwise indicated by an image referred to as a “segmentation mask.” For instance, a segmentation mask can be a binary image having the same dimensions as an original image in which a pixel or voxel is assigned a first value (e.g., 1) if it represents a pixel or voxel in the original image that is part of a segmented region, or is assigned a second value (e.g., 0) if it represents a pixel or voxel in the original image that is not part of the segmented region. In various examples, the image analyzer 122 generates a segmentation mask indicating regions of the image(s) 120 that depict one or more type of non-cancer cells.

Various techniques can be used to segment the image(s) 120. According to some examples, the image analyzer 122 includes a computing model that generates a segmentation mask based on the image(s) 120. For instance, the image analyzer 122 may include a CNN that has been trained to generate a segmentation mask representing non-cancer cells in an input image. U-Net is a type of CNN that is capable of image segmentation tasks (see, e.g., Drioua et al., Sensors (Basel), 23 (17):7318 (2023)). In various cases, the image analyzer 122 generates the segmentation mask by inputting the image(s) 120 into the CNN. Other segmentation techniques are also possible. For example, the image analyzer 122 may generate a segmentation image representing the non-cancer cells in the image(s) 120 via thresholding (e.g., Otsu's method, histogram thresholding, etc.), clustering (e.g., k-means clustering, mean shift method, etc.), edge detection, graph partitioning, or any combination thereof.

In some cases, the image analyzer 122 may identify one or more types of non-cancer cells in the image(s) 120 using image segmentation. In some examples, the image analyzer 122 identifies other types of structures depicted in the image(s) 120, such as the stroma 115, using image segmentation techniques. For instance, the image analyzer 122 may generate a first segmentation mask corresponding to the lymphocytes 112 depicted in the image(s) 120, a second segmentation mask corresponding to the fibroblasts 114 depicted in the image(s) 120, a third segmentation mask corresponding to the stroma 115 depicted in the image(s) 120, a fourth segmentation mask corresponding to the epithelial cells 116 depicted in the image(s) 120, or any combination thereof.

The image analyzer 122 may generate the microenvironment metric(s) 124 based on the identified non-cancer cells depicted in the image(s) 120. In some examples, the image analyzer 122 generates the microenvironment metric(s) 124 by counting the non-cancer cells. For example, the image analyzer 122 may count regions of a segmentation mask that correspond to one or more types of non-cancer cells. In particular instances, the image analyzer 122 counts a number of lymphocytes 112, fibroblasts 114, epithelial cells 116, or any combination thereof, depicted in the image(s) 120. In some cases, the image analyzer 122 determines distances between the non-cancer cells depicted in the image(s) 120. For example, the image analyzer 122 may determine an average distance (e.g., in pixels) between the regions of the segmentation mask. According to some examples, the image analyzer 122 determines a total area (e.g., in pixels) of the non-cancer cells depicted in the image(s) 120. For example, the image analyzer 122 may determine a number of pixels (or another measure of area) indicating the non-cancer cells in the segmentation mask.

According to various cases, the image analyzer 122 determines the microenvironment metric(s) 124 by determining a proximity of the non-cancer cells to the cancer cells 108 in the tissue sample 106. For example, the image analyzer 122 may generate a first segmentation mask indicating the cancer cells 108 in the tissue sample 106, and a second segmentation mask indicating non-cancer cells in the tissue sample 106. In various cases, the image analyzer 122 may generate the microenvironment metric(s) 124 by determining a distance between at least one of the cancer cells 108 and at least one of the non-cancer cells in the tissue sample 106 by comparing the first segmentation mask and the second segmentation mask.

In some examples, the microenvironment metric(s) 124 include a density of at least one type of non-cancer cell in the tissue sample 106. The image analyzer 122 may determine a number and/or amount of non-cancer cells within at least a portion of the tissue sample 106. For instance, the image analyzer 122 may determine a density of the non-cancer cells in the stroma 115 of the tissue sample 106. In some cases, the image analyzer 122 determines a density of the non-cancer cells in the entire portion of the tissue sample 106 depicted by the image(s) 120. In particular cases, the microenvironment metric(s) 124 include a density of the lymphocytes 112 in the tissue sample 106, a density of the fibroblasts 114 in the stroma 115 of the tissue sample 106, an amount or density of epithelial cells 116 in the tissue sample 106, or any combination thereof.

A predictive model 126, according to various implementations, is configured to predict whether the cancer cells 108 are susceptible to one or more treatments based, at least in part, on the microenvironment metric(s) 124. The predictive model 126, for example, may include one or more mathematical and/or computer-based models that are configured to predict the treatment indicator based on the microenvironment metric(s) 124. For instance, the predictive model 126 may include a regression model, threshold rule, confidence interval, or other type of statistical model capable of categorizing the cancer based on the microenvironment metric(s) 124. In various cases, the predictive model 126 includes at least one classifier configured to generate the treatment indicator(s) 128 based on the microenvironment metric(s) 124. In various cases, the predictive model 126 includes one or more ML models that have been pretrained to predict whether the cancer cells 108 are susceptible to the treatment(s).

The predictive model 126 may predict the susceptibility of the cancer cells 108 to various types of treatments. For example, the predictive model 126 may be configured to determine whether the cancer cells 108 are susceptible to a chemotherapy, an immunotherapy, a radiotherapy, a surgical intervention, a cell-based therapy, any other type of therapy described herein, or any combination thereof. In some cases, the predictive model 126 is configured to determine whether the cancer cells 108 are susceptible to a combination therapy that includes two or more types of treatments to be administered to the subject 102.

The predictive model 126 receives input data including the microenvironment metric(s) 124, performs one or more operations based on the input data, and generates output data including one or more treatment indicators 128. The treatment indicator(s) 128, for instance, include data representative of whether the cancer cells 108 are predicted to be susceptible to the one or more treatments. In some examples, the treatment indicator(s) 128 include a likelihood that the cancer cells 108 are susceptible to a given treatment or an indication (e.g., a Boolean value) that there is greater than a threshold (e.g., 90%) likelihood that the cancer cells 108 are susceptible to the given treatment. In some cases, the treatment indicator(s) 128 include a likelihood that the cancer cells 108 are not susceptible (e.g., resistant) to the given treatment or an indication that there is greater than a threshold (e.g., 90%) likelihood that the cancer cells 108 are not susceptible to the given treatment.

The characteristics of the non-cancer cells in the tissue sample 106 may be relevant to predictions of whether the cancer cells 108 are susceptible to various treatments. For example, the cancer cells 108 may be susceptible to an immunotherapy if the lymphocytes 112 are located in the vicinity of the cancer cells 108 within the tissue sample 106. Lymphocytic density within the tissue sample 106, for instance, is correlated to whether an immunotherapy targeting a ligand expressed by the cancer cells 108 will successfully attract lymphocytes 112 in order to destroy the cancer cells 108. By considering the microenvironment metric(s) 124, the predictive model 126 may more accurately predict the efficacy of cancer treatments (e.g., immunotherapies) for the subject 102 than models that rely solely on features of the cancer cells 108.

In some examples, the predictive model 126 may also predict the effectiveness of the treatment(s) based on characteristics of the cancer cells 108. For example, the image(s) 120 may depict the tissue sample 106 stained with an immunostain that targets an antigen. In various cases, the image analyzer 122 may analyze the immunostained depictions of the tissue sample 106 in the image(s) 120 in order to determine whether the cancer cells 108 express the targeted antigen. For instance, the image analyzer 122 may include a CNN trained to identify whether the cancer cells 108 express the antigen. In some cases, the image analyzer 122 may predict that the cancer cells 108 express the antigen by determining that a signal (e.g., an amount of a signal in the image(s) representing light emitted by the immunostain) is greater than a threshold. According to some examples, the image analyzer 122 may generate an expression indicator 130 that represents whether the antigen is present on, in, or otherwise expressed by, the cancer cells 108. The input data for the predictive model 126, in some cases, further includes the expression indicator 130. For instance, the predictive model 126 may determine that there is a greater likelihood that the cancer cells 108 are susceptible to an immunotherapy targeting the antigen if the expression indicator 130 indicates that the cancer cells 108 express the antigen. In some cases, the predictive model 126 may determine that there is a minimal likelihood that the cancer cells 108 are susceptible to the immunotherapy if the expression indicator 130 indicates that the cancer cells 108 do not express the antigen.

In some aspects of the present disclosure, the predictive model 126 generates the treatment indicator(s) 128 based at least in part on characteristics of nucleic acid molecules 132 obtained from the subject 102. In some cases, the nucleic acid molecules 132 are extracted from the tissue sample 106. In some cases, the nucleic acid molecules 132 are extracted from a different sample obtained from the subject 102, such as a fluid biopsy sample.

According to some examples, the nucleic acid molecules 132 include genomic DNA (gDNA). For instance, the nucleic acid molecules 132 include chromosomal DNA that is located in, or extracted from, cells in the tissue sample 106. According to some cases, the DNA is extracted from nuclei and the cells in the tissue sample 106 using mechanical shearing and/or the introduction of a chemical (e.g., a detergent). The DNA may be subsequently isolated from proteins and other cellular materials. In some implementations, the nucleic acid molecules 132 indicate an entire genome of the subject 102 and/or cells in the lesion 104. Thus, a genome of the subject 102 and/or the lesion 104 can be determined by sequencing the DNA in the nucleic acid molecules 132.

In some examples, the nucleic acid molecules 132 include RNA. In some implementations, the nucleic acid molecules 132 include messenger RNA (mRNA), microRNA, non-coding RNA, functional RNA, or any combination thereof. Various RNA in the nucleic acid molecules 132 may be indicative of proteins expressed in the cells of the subject 102 and/or the lesion 104.

In some cases, the tissue sample 106 includes cell-free DNA (cfDNA). The cfDNA, for instance, includes circulating tumor DNA (ctDNA) and/or non-ctDNA. In cases wherein the lesion 104 is a tumor, cancer cells within the lesion 104 will lyse and release the ctDNA into the bloodstream of the subject 102. These cancer cells, for example, include circulating tumor cells (CTCs). Further, other cells additionally release non-ctDNA into the bloodstream of the subject. In general, the cfDNA includes fragments with lengths that are in a range of 1 to 500, 3 to 500, or 100 to 500 bases long. For instance, the cfDNA includes fragments that are about 170 bases long and/or fragments that are about 340 bases long. For example, the cfDNA includes fragments that are 100 to 240 bases long and/or fragments that are 270 to 410 bases long.

A sequencer 134 is configured to generate sequence read data 136 indicating the sequences of the nucleic acid molecules 132. The sequencer 134, for instance, includes one or more devices that are configured to generate the sequence read data 136 by processing at least a portion of the tissue sample 106. In some cases, the nucleic acid molecules 132 are extracted from the tissue sample 106. The extraction can be performed by the sequencer 134, by another device, manually (e.g., by a laboratory technician), or any combination thereof. Any appropriate extraction method known to those of ordinary skill in the art can be utilized.

In various cases, the sequencer 134 is configured to perform one or more processes (e.g., chemical reactions) on the nucleic acid molecules 132 in order to prepare the nucleic acid molecules 132 for sequencing. For instance, the sequencer 134 may ligate adapters onto the nucleic acid molecules 132 and/or amplify the nucleic acid molecules 132, such that numerous copies of the ligated nucleic acid molecules 132 are available for sequencing. Examples of the adapters include, for example, amplification primers, flow cell adapter sequences, substrate adapter sequences, or sample index sequences. The nucleic acid molecules 132 (e.g., the ligated nucleic acid molecules 132) may be amplified by generating multiple copies of the nucleic acid molecules 132 using one or more techniques such as polymerase chain reaction (PCR), a non-PCR amplification technique, or an isothermal amplification technique.

The sequencer 134 may identify the length, position, and identity of the bases in the nucleic acid molecules 132 by sequencing the nucleic acid molecules 132 (e.g., the amplified and/or ligated nucleic acid molecules 132). In various implementations, the sequencer 134 utilizes first-generation sequencing (e.g., Sanger sequencing), second-generation sequencing (e.g., massive parallel sequencing), third-generation sequencing (e.g., nanopore sequencing), or a combination thereof. In some cases, the sequencer 134 is configured to sequence substantially all of the nucleotides of all of the nucleic acid molecules 132 fragments obtained from the tissue sample 106. In some examples, the sequencer 134 is configured to perform targeted sequencing. For instance, the sequencer 134 may determine whether the nucleic acid molecules 132 fragments contain one or more predetermined sequences at one or more genomic locations.

In various cases, the sequencer 134 includes one or more sensors that are configured to detect physical signals (also referred to as “detection signals”) that are indicative of the nucleotide sequences of the nucleic acid molecules 132. The sequencer 134 may perform sequencing-by-synthesis. For example, the sequencer 134 may include one or more optical sensors configured to detect optical signals emitted from fluorescently tagged nucleotide triphosphates (NTPs) that are joined together in a synthesized DNA strand using the ligated nucleic acid molecules 132 as templates. The optical signals detected by the optical sensor(s), for instance, are indicative of the sequences of the nucleic acid molecules 132. The sequencer 134 may perform nanopore sequencing. In various cases, the sequencer 134 includes one or more electrical sensors configured to measure an electrical signal (e.g., an electrical current) across a substrate as the ligated nucleic acid molecules 132 are directed through a nanopore extending through the substrate. The electrical signal over time, in various cases, is indicative of the sequences of the nucleic acid molecules 132 in the tissue sample 106. The sequencer 134, in various implementations, is configured to generate the sequence read data 136 as digital data based on the analog signals detected by the sensor(s). For instance, the sequencer 134 includes one or more analog to digital converters (ADCs). In various cases, the sequencer 134 includes at least one processor configured to generate the sequence read data 136.

In some implementations, the sequencer 134 performs RNA sequencing (RNA-seq) on the nucleic acid molecules 132. For example, the nucleic acid molecules 132 include RNA that is extracted from the tissue sample 106. In some examples, the RNA in the nucleic acid molecules 132 is fragmented. In various implementations, complementary DNA (cDNA) is generated using reverse transcriptase, such that the cDNA includes sequences that are complementary to the RNA in the nucleic acid molecules 132 from the tissue sample 106. The cDNA, according to various cases, can be sequenced using the DNA sequencing techniques described above. Accordingly, in some cases, the sequence read data 136 indicates sequences of RNA present in the tissue sample 106, which may be indicative of the transcriptome of the subject 102 and/or the lesion 104.

In various cases, the sequencer 134 performs sequencing on a subset of the nucleic acid molecules 132. For instance, the sequencer 134 may perform targeted sequencing on one or more predetermined genes, such as any of the genes described herein. The sequencer 134, in some cases, may refrain from sequencing at least a portion of the nucleic acid molecules 132 that do not correspond to the subset.

A genomic analyzer 138 identifies genomic features 140 of the nucleic acid molecules 132 by analyzing the sequence read data 136. In various implementations, the genomic analyzer 138 identifies, calculates, or otherwise determines the genomic features 140 based on the sequences of the nucleic acid molecules 132 indicated in the sequence read data 136. One or more types of features are identified by the genomic analyzer 138. The genomic features 140 may be derived from the sequence read data 136.

In some cases, the genomic features 140 include a mismatch repair deficiency (MMRD) probability score. In various cases, the MMRD probability score indicates a likelihood that one or more MMR pathways of cells in the tissue sample 106 are ineffective at performing mismatch repair. In some implementations, the MMRD probability score is determined by determining genomic features by analyzing the sequence read data 136, inputting the genomic features into at least one trained machine learning model trained to generate the MMRD probability score based on previously analyzed data from a population omitting the subject 102. The genomic features relevant to the MMRD probability score include, for instance, a fraction unstable score, a composite COSMIC single-base substitution signature, a COSMIC indel signature, a copy number signature, a tumor mutational burden score, a blood-based tumor mutational burden score, a germline status for a mutation in one or more genes associated with DNA mismatch repair (MMR) (also referred to as “MMR genes”), a methylation status for the one or more MMR genes, a methylation status for one or more promoters associated with the one or more MMR genes, a methylation status of one or more enhancers associated with the one or more MMR genes, or any combination thereof. Examples of the MMR genes include, for instance, MSH2, MSH6, PMS2, or MLH1.

The genomic features 140, in some examples, include a copy number state of one or more genetic loci indicated by the sequence read data 136. In various implementations, a number of copies of a predetermined sequence at a given locus in the genome of the subject 102 and/or the lesion 104 (also referred to as a “copy number” of the locus) is determined. The copy number state, in various implementations, may indicate copy numbers of one or more loci in the genome of the subject 102 and/or the lesion 104. For instance, the copy number state may indicate the presence and/or amount of copies of various sequences present in the genome of the subject 102 and/or the lesion 104, which may be due to copy number variation.

According to various examples, the sequence read data 136 may represent a genome of the subject 102 and/or the lesion 104. Various portions of the sequence read data 136 are aligned with at least one reference sequence (e.g., a reference genome). The aligned data is segmented using at least one segmentation technique (e.g., a circular binary segmentation (CBS) method, a maximum likelihood method, a hidden Markov chain method, a walking Markov method, a Bayesian methods, a long-range correlation method, a change point method, or any combination thereof), thereby generating non-overlapping segments of the sequence read data 136, wherein a sequence associated with a given segment is associated with the same copy number (e.g., a number of instances in which the sequence appears in the segment). Various genetic loci are binned, or otherwise sorted, with respect to the segments of the genome of the subject 102 and/or the lesion 104. The copy number state, for instance, is representative of the respective copy numbers associated with the genetic loci.

In some implementations, the genomic features 140 include the presence or absence of a variant (e.g., a pathogenic variant) in one or more genes associated with classifying the lesion 104. In various cases, the genes include one or more of ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSFIR, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, L1CAM, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKC1, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, or ZNF703. In some cases, the genes include one or more of ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1B, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3Kδ, PIGF, PTCH, RAF, RANKL, RET, ROS1, SLAMF7, VEGF, VEGFA, or VEGFB. In some examples, the genes include an estrogen receptor (ER) gene and/or a progesterone receptor (PR) gene.

In some cases, the genomic features 140 are indicative of microsatellite instability (MSI). Microsatellites are highly polymorphic DNA-repeat regions. In certain examples, “microsatellite” refers to a repetitive nucleic acid having repeat units of less than about 10 base pairs or nucleotides in length. In certain examples, a microsatellite refers to a tract of tandemly repeated (i.e., adjacent) DNA motifs ranging from one to six or up to ten nucleotides, with each motif repeated 5 to 50 repeated times. During DNA replication, mutations (e.g., insertions or deletions) are more likely to be introduced at microsatellites than various other portions of the genome. In various cases, these mutations are corrected via MMR pathways. However, if the MMR pathways are impaired (e.g., the MMR genes of the hosting cell include variants that impede function), then the mutations at the microsatellites may be substantially retained. “Microsatellite instability” refers to genetic instability in the microsatellite regions. Cancer patients with microsatellite instability classified as being high (MSI-H or MSI-High) frequently exhibit an accumulation of somatic mutations in tumor cells that leads to a range of molecular and biological changes including high tumor mutational burden, increased expression of neoantigens and abundant tumor-infiltrating lymphocytes. Chang et al. “Microsatellite Instability: A Predictive Biomarker for Cancer Immunotherapy,” Appl Immunohistochem Mol Morphol, 26(2):e15-e21 (2018). These changes have been linked to increased sensitivity to checkpoint inhibitor drugs, such as pembrolizumab, which is used to treat advanced melanoma, head and neck squamous cell carcinoma, non-small cell lung cancer (NSCLC), and classical Hodgkin lymphoma. According to various examples, “MSI score” refers to an amount of instability in one or more microsatellites. For example, an MSI score can be represented as a fraction (i.e., an “MSI fraction”) of instability in the one or more microsatellites. Other types of portions of DNA may be associated with a high likelihood of mutations. In some cases, the genomic features 140 include a fraction unstable score, indicative of mutations in the microsatellites and other portions of the genome that are prone to mutations.

In various cases, an MSI score can be determined based on a predetermined set of repetitive loci (e.g., 2000 repetitive loci, each with a minimum of 5 repeat units of mono-, di-, and trinucleotides). By evaluating the sequence read data 136, the genomic analyzer 138 may determine lengths of repetitive sequences corresponding to the loci. If an example locus among the loci corresponds to a predetermined repeat length, the locus is considered to be “unstable.” The MSI score, for instance, is determined by determining an amount of the unstable loci (e.g., a fraction of the unstable loci with respect to the total number of repetitive loci evaluated). In some cases, the MSI score is used to determine whether the subject 102 and/or lesion 104 is MSI-High (MSI-H). For example, MSI-H status may be applicable if the MSI score is greater than a threshold (e.g., 0.5%). Techniques for determining MSI scores are described, for instance, in Woodhouse et al., “Clinical and analytical validation of FoundationOne LiquidCDx, a novel 324-Gene cfDNA-based comprehensive genomic profiling assay for cancers of solid tumor origin,” PLOS ONE 15 (9) (2020).

In some implementations, the genomic features 140 include a mutation signature. In various cases, a mutational signature can represent an amount and/or identity of mutations (e.g., insertions, deletions, double-base substitutions, single-base substitutions, or any combination thereof) indicated in the nucleic acid molecules 132 from the subject 102. In some cases, the mutational signature indicates an amount (e.g., number or percentage) of individual classes of base substitutions present in the nucleic acid molecules 132. For instance, the classes include single-base substitutions including C>A, C>G, C>T, T>A, T>C, and T>G. A mutational signature can be derived by comparing the sequences indicated in the sequence read data 136 to at least one reference sequence, such as a reference genome. For example, the genomic features 140 may include a Catalogue Of Somatic Mutations In Cancer (COSMIC) mutational signature, such as a COSMIC indel signature. In some cases, the genomic features 140 include a single-base substitution signature. According to some cases, the mutation signature is derived based on a model, such as an autoencoder model.

In various examples, the genomic features 140 include a tumor mutational burden (TMB) score. The TMB, for instance, is a measure of the number of mutations carried by the cancer cells 108. By comparing DNA sequences from a patient's healthy tissues and cancer cells, the number of acquired somatic mutations present in tumors, but not in normal tissues, may be determined. In some instances, driver mutations may be excluded from a TMB calculation. In certain examples, “tumor mutational burden” or “TMB score” refers to the number of somatic mutations in a tumor's genome and/or the number of somatic mutations per area of the tumor's genome. In some embodiments, TMB, as used herein, refers to the number of somatic mutations per megabase (Mb) of DNA sequenced. In some embodiments, germline (inherited) variants are excluded when determining TMB, given that the immune system has a higher likelihood of recognizing these as self. In addition, germline variants do not reflect the biology of somatic mutation for the purposes of TMB determinations. In various cases, driver mutations are excluded from a TMB calculation.

In some cases, the genomic features 140 include the presence, amount, type, or any combination thereof, of one or more hotspot mutations in the nucleic acid molecules 132. Hotspots, for instance, can refer to loci in the genome of the subject 102 and/or the lesion 104 that are prone to mutation. Examples of hotspots include CpG islands, microsatellites, centromeric DNA, telomers, subtelomeric regions, common fragile sites, palindromic AT-rich repeats (PATRRs), G-quadruplexes, R-loops, and the like.

Hotspot mutations give rise to oncological outcomes. PhyloP, SIFT, Grantham, COSMIC and PolyPhen-2 are in silico tools that can be used to assess pathogenicity of identified variants. Exemplary hotspot genes and mutations include EGFR exon 19 activating mutation, EGFR exon 19 deletion, EGFR exon 19 insertion, EGFR exon 19 sensitizing mutation, EGFR exon 20 activation mutation, EGFR exon 20 insertion, EGFR G719 mutation, EGFR L858R mutation, EGFR L861 mutation, EGFR S768 mutation, EGFR T790M mutation, C797 mutation, KIT activating mutation, KRAS activating mutation, MET activating mutation, NRAS activating mutation, PMS2 promoter mutations, among many others. Hotspot mutations also occur in the following genes: AKT2, BRCA1, BRCA2, ERC1, NSD1, POLH, PPM1G, PTEN, RAD18, RAD51, RAD51B, RB1, TERT, TP53, TP53Bp1, ALK, ARMT1, ATAD5, ATG7, ATIC, AXL, BIRC6, BRD3, BRD4, CAPRIN1, CCAR2, CCDC6, CDK5RAP2, CHD9, CIT, CTNNB1, CUL1, EBF1, EIF3E, HIP1, HMGA2, IRF2BP2, NOTCH1, NOTCH4, NPM1, OFD1, TACC1, TACC3, TERF2, TMEM106B, UBE2L3, USP10, WRDR48, YAP1, ZEB2, and ZMYND8.

The genomic features 140, in particular examples, include the presence, amount, type, or any combination thereof, of one or more aneuploidy events. For instance, the genomic features 140 may indicate whether the subject 102 and/or the lesion 104 includes one or more extra chromosomes (e.g., greater than a pair of 23 chromosomes) or one or more missing chromosomes (e.g., less than the pair of 23 chromosomes).

In some implementations, the genomic features 140 include a tumor purity of the tissue sample 106. In various implementations, the tumor purity represents an amount of the nucleic acid molecules 132 that originate from a tumor (e.g., the cancer cells 108) with respect to a total amount of the nucleic acid molecules 132 in the tissue sample 106. Tumor purity can be estimated, for instance, based on a presence or amount of somatic copy-number alterations (SCNA), single-nucleotide variants (SNVs), minor allele frequency (MAF), or any combination thereof, observed with respect to the sequence read data 136.

In various examples, the genomic features 140 are included in the input data provided to the predictive model 126. The predictive model 126 may determine the treatment indicator(s) 128 based at least in part on the genomic features 140.

A report generator 142 is configured to generate a report 144 based, at least in part, on the treatment indicator(s) 128. The report 144, for example, includes consumable data that can inform the care provider 105 about the treatment indicator(s) 128 of the subject 102. In various implementations, the report 144 may indicate the results of additional analyses, such as the results of a histological study, whole transcriptome sequencing, cfRNA sequencing, whole exome sequencing, whole genome sequencing, a cancer (e.g., DNA) hotspot panel test, a DNA methylation test, a tumor mutational burden (TMB) test, a DNA fragmentation test, an RNA fragmentation test, a microsatellite instability (MSI) test, a tumor mutational burden (TMB) test, or a viral status test. The performance of such tests is within the ordinary skill of the art, with additional detail provided elsewhere herein. The report 144, for example, may include a genomic profile of the subject 102 based on various combinations of the above analyses and tests.

In some implementations, the report 144 indicates that a follow-up test of the subject 102 is indicated. For instance, in response to determining that the categorization of the disease is inconclusive, the report generator 142 may generate the report 144 to indicate that one or more additional tests (e.g., a histological study, genome sequencing, exome sequencing, additional DNA sequencing, RNA sequencing, transcriptome sequencing, etc.) should be performed in order to identify the cancer of the subject 102.

In various cases, the report 144 is output to a clinical device 146. For example, the report generator 142 transmits the report 144 to the clinical device 146. In various implementations, the clinical device 146 is a computing device that is operated by, owned by, or otherwise associated with the care provider 105. For instance, the clinical device 146 may be a desktop computer, a laptop computer, a smart phone, or some other computing device associated with the care provider 105. The clinical device 146, in various cases, outputs the report 144 to the care provider 105. In some cases, the clinical device 146 includes a display (e.g., a screen) that visually presents the report 144. In various cases, the clinical device 146 includes a speaker that outputs a sound indicative of the report 144. The clinical device 146, in various cases, may output the information in the report 144 using one or more output mechanisms or devices.

The care provider 105 may review the report 144 by interacting with the clinical device 146. The report 144, in various cases, may enhance the clinical decision-making of the care provider 105. For instance, the care provider 105 may prepare and/or administer a treatment to the subject 102 based on the report 144. According to various implementations, the care provider 105 may initiate the treatment and/or refer the subject 102 to another care provider to receive the treatment. In various cases, if the disease of the subject 102 is cancer, the care provider 105 may prescribe, recommend, or administer an anticancer agent for the subject 102. For example, the care provider 105 may rely on the treatment indicator(s) 128 reflected in the report 144 to select a treatment that the cancer cells 108 are predicted to be susceptible to.

In various implementations, the care provider 105 may develop a diagnosis and/or prognosis of the subject 102 based on the report 144. In various implementations, the care provider 105 may communicate information in the report 144 to the subject 102.

FIG. 1 illustrates various elements that can be embodied in one or more computing devices. For example, at least a portion of the functions of the imaging device 118, the image analyzer 122, the predictive model 126, the sequencer 134, the genomic analyzer 138, the report generator 142, the clinical device 146, or any combination thereof, are performed by one or more processors in at least one computing device. Examples of computing devices include server computers, desktop computers, laptop computers, tablet computers, mobile phones, wearable devices, Internet of Things (IoT) devices, and the like. In various cases, instructions for performing at least a portion of the functions of these elements are stored in memory and/or in a non-transitory computer readable medium. The instructions, for instance, are executed by the processor(s).

FIG. 1 also illustrates various types of data. For example, the image(s) 120, the microenvironment metric(s) 124, the treatment indicator(s) 128, the expression indicator 130, the sequence read data 136, the genomic features 140, the report 144, or any combination thereof, includes data. The various types of data illustrated in FIG. 1 may be stored, such as in memory or in non-transitory computer readable media. In various implementations, at least a portion of the data is transmitted or otherwise output by one or more computing devices. For example, a computing device may transmit one or more communication signals to another computing device, wherein the communication signal(s) encode at least a portion of the data. Examples of communication signals include electromagnetic signals, optical signals, ultrasonic signals, optical signals, and electrical signals. For example, communication signals can be transmitted wirelessly and/or in a wired fashion. The communication signals, for instance, are transmitted over one or more wireless channels and/or one or more wired channels (e.g., optical cabling, electrical cabling, etc.). In various cases, the communication signal(s) are transmitted over one or more communication networks. A communication network, for instance, may be defined according to one or more physical channels, such as one or more frequency spectra. In some cases, a communication network is defined according to one or more communication protocols and/or standards. Examples of communication networks include fiber optic networks, Institute of Electrical and Electronics Engineers (IEEE) networks (e.g., WI-FI™ networks, WiMAX networks, BLUETOOTH™ networks, etc.), cellular networks (e.g., a 3rd Generation Partnership Project (3GPP) radio network, such as a Long Term Evolution (LTE) network, a New Radio (NR) network; or a cellular core network such as a 3rd Generation (3G) core, a 4th Generation (4G) core, a 5th Generation (5G) core, etc.), ultrasonic networks, and the like. In some cases, the data is broadcasted from one device to multiple other devices. In some cases, the data is unicasted from one device to another device. For instance, various forms of data described herein may be transmitted via a peer-to-peer (P2P) connection.

A particular example will now be described with reference to FIG. 1. In this example, the subject 102 presents to a clinical environment with symptoms of lung cancer. The care provider 105 initiates medical imaging (e.g., x-ray imaging) on the subject 102 and discovers the lesion 104 located on a lung of the subject 102. The care provider 105 obtains the tissue sample 106, for instance, by performing a needle biopsy procedure on the lesion 104.

In various cases, the nucleic acid molecules 132 are extracted from a portion of the tissue sample 106. A remaining portion of the tissue sample 106 is treated with a fixation agent and divided into slices. At a first stage, a slice of the tissue sample 106 is treated with H&E. The imaging device 118 generates a first image among the image(s) 120 of the H&E-stained tissue sample 106. Subsequently, the slice of the tissue sample 106 may be washed and treated with an immunostain. According to some examples, the imaging device 118 generates a second image among the image(s) 120 of the immunostained tissue sample 106. In various cases, the immunostain specifically binds PD-L1.

The image analyzer 122 generates the microenvironment metric(s) 124 based on the first image among the image(s) 120. Specifically, the image analyzer 122 generates a first segmentation mask representing the lymphocytes 112 depicted in the first image. In various cases, the image analyzer 122 determines a density of the lymphocytes 112 (e.g., number of the lymphocytes 112 in the area represented by the first image) by counting the shapes indicating the lymphocytes in the first segmentation mask. In various cases, the image analyzer 122 generates a second segmentation mask representing the fibroblasts 114 in the stroma 115 depicted in the first image. The image analyzer 122, for instance, determines a density of the fibroblasts 114 in the stroma 115 (e.g., number of the fibroblasts in the area of the first image depicting the stroma 115) by counting the shapes indicating the fibroblasts 114 in the second segmentation mask. In some examples, the image analyzer 122 further determines a metric indicative of the epithelial cells 116 in the tissue sample 106. For example, the image analyzer 122 may determine an average distance between the epithelial cells 116 and the cancer cells 108 by generating a third segmentation mask corresponding to the cancer cells 108 depicted in the first image, generating a fourth segmentation mask corresponding to the epithelial cells 116 depicted in the first image, and comparing the third and fourth segmentation masks. The density of the lymphocytes 112, the density of the fibroblasts 114 in the stroma 115, and the average distance between the epithelial cells 116 and the cancer cells 108, for instance, are included in the microenvironment metric(s) 124.

In various examples, the image analyzer 122 further determines whether the cancer cells 108 express PD-L1 by analyzing the second image depicting the immunostained tissue sample 106. According to various cases, the immunostain is a fluorescent immunostain that emits light having a first wavelength in response to receiving light having a second wavelength. In various cases, the imaging device 118 emits the light having the second wavelength toward the immunostained tissue sample 10 in order to obtain the second image. In various cases, the image analyzer 122 generates a segmentation mask representing the cancer cells 108 in the second image. The image analyzer 122 may extract the portion of the second image that is representative of the cancer cells 108 by performing pixel-by-pixel multiplication of the second image and the segmentation mask. In various cases, the image analyzer 122 may assess the expression of PD-L1 based on assessing an amount of pixels in the portion of the second image that have a color associated with the light having the first wavelength. In some cases, the image analyzer 122 quantifies the amount of PD-L1 expression based on the number of pixels depicting the cancer cells 108 that depict the light emitted by the immunostain. In some cases, the expression indicator 130 indicates the presence or amount of PD-L1 expression of the cancer cells 108.

Additionally, the extracted nucleic acid molecules 132 are sequenced by the sequencer 134. In various cases, the genomic features 140 identified by the genomic analyzer 138 include MSI score and TMB.

The microenvironment metric(s) 124, the expression indicator 130, and the genomic features 140 are included in input data provided to the predictive model 126. In various cases, the predictive model 126 determines whether the cancer cells 108 are susceptible an immunotherapy targeting PD-L1 (e.g., pembrolizumab) based on the input data. For example, the predictive model 126 may determine that the cancer cells 108 express PD-L1 based on the expression indicator 130 and the genomic features 140, but that the microenvironment metric(s) 124 indicate that the tumor microenvironment of the subject 102 is not conducive to targeting the cancer cells 108 using an immunotherapy. For example, the density of the lymphocytes 112 may be below a predetermined threshold. Accordingly, the predictive model 126 may indicate, in the treatment indicator(s) 128, that the cancer cells 108 are predicted to be resistant to treatment using the immunotherapy targeting PD-L1, despite the fact that the cancer cells 108 express PD-L1. In some cases, the predictive model 126 outputs the treatment indicator(s) 128 based on a more sophisticated analysis of various characteristics of the microenvironment metric(s) 124, the expression indicator 130, and the genomic features 140.

Accordingly, the report generator 142 may generate the report 144 to indicate a recommendation against administering the immunotherapy to the subject 102 and/or to indicate a recommendation for administering an alternative treatment (e.g., surgery, a chemotherapy, or the like) to the subject 102. Upon reviewing the report 144 on the clinical device 146, the care provider 105, in some cases, administers the alternative treatment. Accordingly, the subject 102 may be prevented from experiencing side effects of the immunotherapy without the immunotherapy treating the cancer cells 108 in the lesion 104.

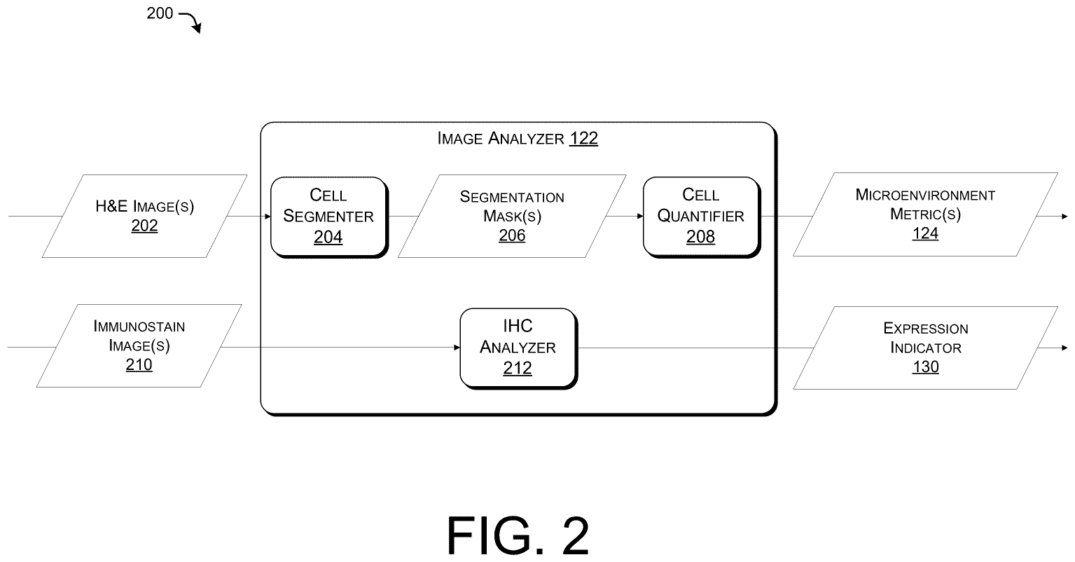

FIG. 2 illustrates an environment 200 with an example of the image analyzer 122 described above with reference to FIG. 1.

The image analyzer 122 receives one or more H&E images 202. The H&E image(s) 202, for instance, are included in the image(s) 120 described above with reference to FIG. 1. In various implementations, the H&E image(s) 202 depict one or more stained slices of a tissue sample obtained from a subject. The tissue sample, for instance, is a sample from a tumor or a potentially cancerous lesion.

A cell segmenter 204 is configured to generate one or more segmentation masks 206 based on the H&E image(s) 202. The segmentation mask(s) 206, in various cases, indicate one or more types of non-cancer cells depicted in the H&E image(s) 202. In some implementations, the cell segmenter 204 includes one or more CNNs configured to segment the non-cancer cells depicted in the H&E image(s) 202. In some cases, the cell segmenter 204 performs other types of segmentation techniques in order to generate the segmentation mask(s) 206. The segmentation mask(s) 206, for instance, have the same pixel dimensions as the H&E image(s) 202. An example pixel in the segmentation masks(s) 206 corresponds to an example pixel at the same row and column in the H&E image(s) 202, for instance. In some examples, the segmentation masks 206 include one or more binary images whose pixels corresponding to a non-cancer cell depicted in the H&E image(s) 202 have a first value (e.g., 1 or 255), and whose pixels that do not correspond to any non-cancer cell depicted in the H&E image(s) 202 have a second value (e.g., 0). In some examples, the segmentation masks 206 have some of the pixels of the H&E image(s) 202 corresponding to regions that depict one or more types of non-cancer cells.

A cell quantifier 208 is configured to generate the microenvironment metric(s) 124 based on the segmentation mask(s) 206. For example, the cell quantifier 208 may be configured to count regions indicated in the segmentation mask(s) 206, distances between regions indicated in the segmentation mask(s) 206, an amount of pixels indicating the non-cancer cells in the segmentation masks(s) 206, or any combination thereof.

In various cases, the image analyzer 122 additionally receives one or more immunostain images 210. The immunostain image(s) 210, in some cases, are included in the image(s) 120. The immunostain image(s) 210 depict one or more slices of the tissue sample when the tissue sample has been stained with at least one immunostain. In various cases, the immunostain includes a fluorescent tag and the immunostain image(s) 210 are obtained by illuminating the stained tissue sample with excitation light, thereby causing the immunostain to fluoresce when the immunostain image(s) 210 are obtained. In various cases, the immunostain image(s) 210 indicates whether one or more cancer cells in the tissue sample express one or more antigens-of-interest.

An IHC analyzer 212 is configured to generate the expression indicator 130 based on the immunostain image(s) 210. The expression indicator 130, for instance, includes data indicating whether cells (e.g., the cancer cells) depicted in the immunostain image(s) 210 express the antigen(s)-of-interest. In various cases, the IHC analyzer 212 includes one or more computing models that detect expression of the antigen(s)-of-interest based on the immunostain image(s) 210. For instance, the IHC analyzer 212 may include a first computing model (e.g., a trained CNN) configured to generate a segmentation map indicating the cancer cells depicted in the immunostain image(s) 210, and also a second computing model (e.g., a thresholding model) configured to determine whether the cancer cells indicated by the segmentation map include greater than a threshold number of pixels corresponding to the color of the immunostain.

In various implementations of the present disclosure, a computing model (e.g., the predictive model 126) determines whether the cancer cells in the tissue sample, and whether other cancer cells present in the body of the subject from which the tissue sample is obtained, are susceptible to one or more treatments based on the microenvironment metric(s) 124. In various cases, the computing model further determines whether the cancer cells are susceptible to the treatment(s) based on the expression indicator 130 and/or genomic features of the tissue sample (or another sample obtained from the subject).

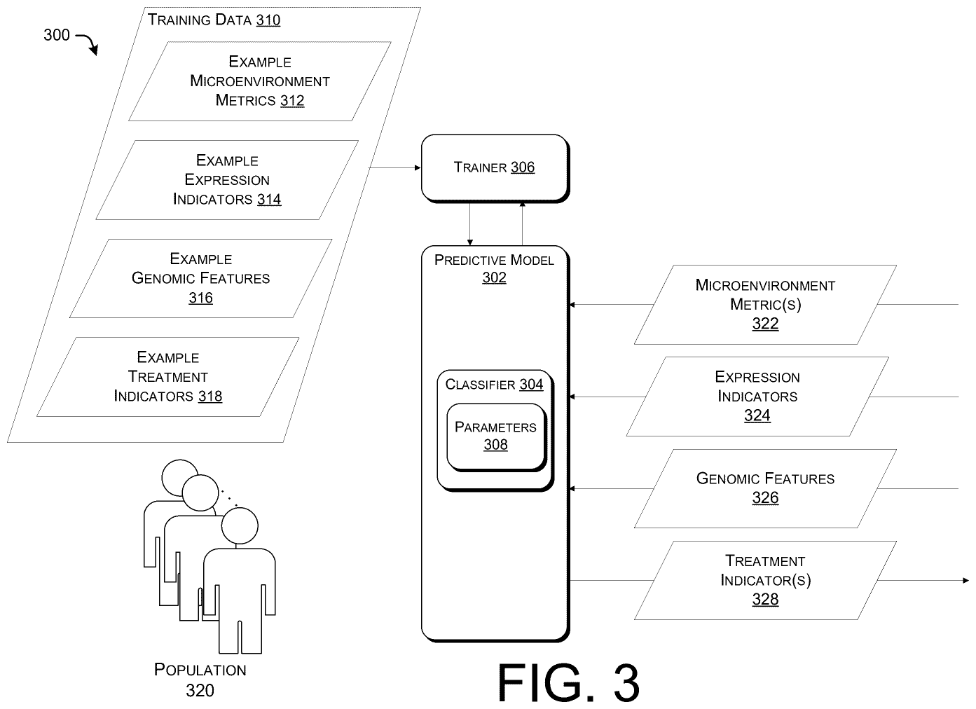

FIG. 3 illustrates an example environment 300 for training and utilizing a predictive model 302 to predict whether an anticancer treatment will be effective when administered to a subject. The predictive model 302, for instance, is the predictive model 126 described above with reference to FIG. 1. In various implementations, the predictive model 302 includes a classifier 304, which may include one or more ML models. A trainer 306, for instance, is configured to optimize various parameters 308 of the classifier 304 based on training data 310.

The training data 310 includes example microenvironment metrics 312, example expression indicators 314, example genomic features 316, and example treatment indicators 318. The example microenvironment metrics 312, in various cases, are obtained based on histological images (e.g., images of H&E stained tissue samples) of tissue samples obtained from individuals in a population 320. The example expression indicators 314 may include data indicating whether cells (e.g., cancer cells) in the tissue samples express one or more antigens-of-interest. The example genomic features 316, for instance, may include genomic features of individuals in the population 320. The example treatment indicators 318, in various cases, may include ground truth efficacy of one or more treatments in addressing cancer cells of the individuals within the population 320. For example, the example treatment indicators 318 may indicate whether one or more treatments (e.g., at least one immunotherapy targeting the antigen(s)-of-interest, a chemotherapy, a combination therapy, a surgical intervention, radiotherapy, etc.) reduced a number of cancer cells in the population 320, increased survivability of cancers experienced by the population 320, other prognostic indicators of the individuals in the population 320, or the like. In some cases, the treatment indicators 318 provide survivability and/or survivorship information about the individuals of the population 320, such as whether the individuals survived up to a particular time period after receiving an initial diagnosis of cancer. In some cases, the example treatment indicators 318 are generated based on clinical analyses of the individuals within the population 320 after the treatment(s) are administered.

The classifier 304, for instance, may include one or more model types. For instance, the classifier 304 include an artificial neural network. An artificial neural network includes various layers that respectively process input data. For example, an artificial neural network includes an input layer, one or more hidden layers, and an output layer. The input layer performs a pre-processing operation on the input data. The hidden layer(s) may perform various processing operations on the output from the input layer. The output layer, in various cases, processes the output from the hidden layer(s). Each layer, in some cases, includes one or more nodes, which are defined by individual operations. In various cases, the hidden layer(s) include nodes that are connected to each other in parallel and/or series. Examples of artificial neural networks include feedforward neural networks, multi-layer perceptrons (MLPs), CNNs, and backpropagation models. In various implementations, the operations performed by the layers and/or nodes within an artificial neural network included in the classifier 304 is defined according to the parameters 308. For example, the parameters 308 may include weights, thresholds, filters, kernels, or other data objects that are utilized to perform operations of the classifier 304.

In some implementations, the classifier 304 include a nearest-neighbor model. One example of a nearest-neighbor model includes a k-nearest neighbor model. For example, a nearest-neighbor model defines various “neighbors,” which are points within a feature space, with associated class labels. When a new data point is mapped to the feature space, the new data point is classified based on the proximity (e.g., Euclidian distance, Manhattan distance, Minkowski distance, etc.) of its “neighbors” to the new data point as well as their associated classes. In some cases, the new data point is classified as belonging to a particular class if greater than a threshold number of neighbors within a threshold distance of the new data point are members of the class. For instance, the parameters 308 may include k (e.g., the number of neighbors compared to the new data point), the threshold distance, and so on.

In various cases, the classifier 304 include a regression analysis model. The regression analysis model, for example, is defined by a regression function that defines relationships between one or more independent variables and one or more dependent variables. The regression function may further define one or more unknown parameters that define a relationship between the independent and dependent variables. In various implementations, the unknown parameters and/or the type of regression function (e.g., linear, quadratic, etc.), is defined according to the parameters 308.

In some cases, the classifier 304 include a clustering model. In various cases, a clustering model maps various data points (e.g., training data) to a feature space. Based on the proximity of groups of those data points in the features pace, one or more “clusters” are defined. An additional data point may be classified according to one or more of the clusters based on its proximity to the clusters (e.g., a center of the clusters, a boundary of the cluster, etc.). Examples of clustering models include k-means clustering, mean-shift clustering, expectation-maximization (EM) clustering, and agglomerative hierarchical clustering. The parameter(s) 308, for example, include a threshold proximity within which a new data point is classified within a cluster, a density of points used to define a cluster, and the like.

In various examples, the classifier 304 include a principal component analysis model. In various implementations, a principal component analysis defines a collection principal components of unit vectors within a coordinate space based on a data set (e.g., training data). The model, for example, is an orthogonal linear transformation of the data set. Various weights of the model, for example, are included in the parameter(s) 308.

The classifier 304, in some implementations, includes a gradient boosting model. For example, the gradient boosting model is defined as a collection of prediction models (e.g., decision trees) that iteratively classify observed data. In various cases, the type of prediction model, weights in the prediction models, and the like, are defined by the parameter(s) 308.

The classifier 304, for example, includes a random forest. The random forest, for instance, includes multiple decision trees that classify data in an ensemble fashion. In various implementations, the decision trees are defined by the parameter(s) 308.

In various implementations of the present disclosure, the trainer 306 is configured to optimize the parameters 308 of the classifier 304 based on the training data 310. For example, the trainer 306 may input first example features (corresponding to a first individual among the population 320) among the example microenvironment metrics 312, the example expression indicators 314, and the example genomic features 316, into the predictive model 302, and may receive a predicted treatment indicator. The trainer 306 may compute a loss (e.g., determine a discrepancy) between a first example treatment indicator (corresponding to the first individual) among the example treatment indicators 318 and the predicted treatment indicator. Further, the trainer 306 may alter the parameters 308 in order to minimize the loss. In various cases, the trainer 306 optimizes the parameters 308 iteratively based on the entire set of the training data 310.

In various implementations, the optimization of the parameters 308 enables the predictive model 302 to identify predictive attributes of the example microenvironment metrics 312, the example expression indicators 314, and the example genomic features 316 that are correlated to or otherwise associated with the example treatment indicators 318. For instance, the predictive model 302 may determine that a particular lymphocytic density and antigen expression are associated with a particular immunotherapy being particularly effective at treating cancer cells. The predictive model 302 may therefore classify cancers based on features outside of the example microenvironment metrics 312, example expression indicators 314, and example genomic features 316 by recognizing or otherwise identifying the predictive attributes.

Once the parameters 308 are optimized, the predictive model 302 may be ready to classify a new set of data. For example, the predictive model 302 may receive input data including features of a subject outside of the population 320. These features include, for instance, one or more microenvironment metrics 322, an expression indicator 324, and genomic features 326 of the subject. The features, for instance, may include one or more of the predictive attributes. The predictive model 302 may perform various operations on the input data based on the trained classifier 304 and the optimized parameters 308. In various cases, the predictive model 302 outputs output data including one or more treatment indicators 328 based on the features of the subject. The treatment indicator(s) 328, for instance, indicate whether cancer cells of the subject are susceptible to one or more treatments.

Although FIG. 3 is primarily described as referring to supervised learning, implementations are not so limited. In various cases, the training data 310 omits the example treatment indicators 318 and the trainer 306 is configured to optimize the parameters 308 using the example microenvironment metrics 312, the example expression indicators 314, and the example genomic features 316 and an unsupervised learning technique.

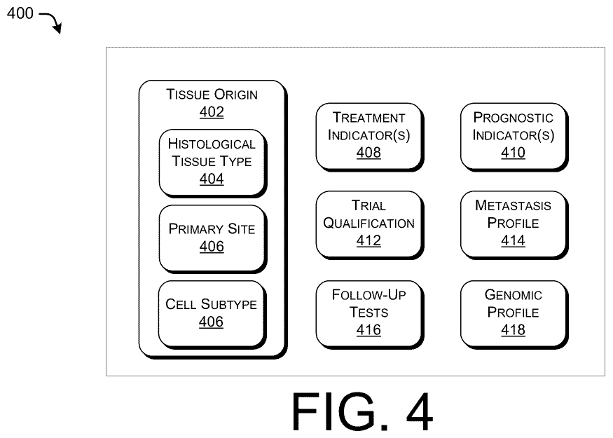

FIG. 4 illustrates an example report 400 of a cancer of a subject. In various cases, the report 400 is the report 144 described above with reference to FIG. 1. The report 400, for instance, may be displayed to a patient and/or care provider. In some cases, the report 400 is generated based on features of a sample (e.g., a tissue sample, a liquid biopsy sample, etc.) obtained from the subject.

The report 400 includes a tissue origin 402 of the cancer. The tissue origin 402, for instance, indicates a histological tissue type 404, a primary site 406, cell subtype 407, or any combination, of the cancer.

In various cases, the report 400 includes one or more treatment indicators 408. For instance, the treatment indicator(s) 408 convey whether the cancer is predicted to be resistant to one or more predetermined therapies and/or whether the cancer is predicted to be responsive to one or more predetermined therapies. In various cases, the treatment indicator(s) 408 provide whether cancer cells of the subject are susceptible (or resistant to) one or more types of treatments.

In some examples, the report 400 includes one or more prognostic indicators 410. The prognostic indicator(s) 410, for instance, indicate a prognosis of the subject in view of the cancer of the subject. For example, the prognostic indicator(s) 410 may indicate a survivability, a recoverability, a quality of life indicator, or other information indicative of the prognosis of the subject.

The report 400 may include a trial qualification 412 of the subject. The trial qualification 412, for instance, indicates whether the subject is predicted to qualify for a predetermined clinical trial.

The report 400, in 4arious implementations, includes a metastasis profile 414 of the subject. The metastasis profile 414, for instance, indicates a likelihood that the cancer will metastasize (e.g., at a particular point in time), one or more tissues in which the cancer is predicted to metastasize, or the like.

In various cases, the report 400 includes recommended follow-up tests 416. For example, the report 400 may include a recommendation to perform whole genome sequencing on the subject, particularly in cases if the cancer cannot be categorized with results from previously performed tests.

The report 400 may include a genomic profile 418 of the subject. In various cases, the genomic profile 418 includes or is generated based on the results of non-fragmentomic analyses of the subject.

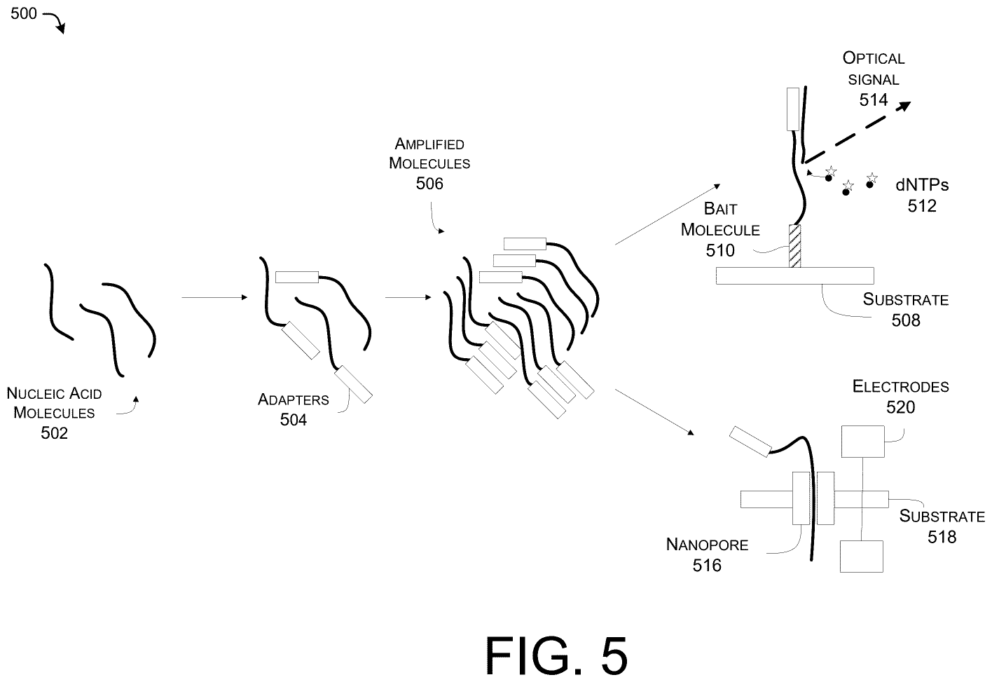

FIG. 5 illustrates an example environment 500 for sequencing various nucleic acid molecules 502. In various implementations, the nucleic acid molecules 502 include cfDNA and/or gDNA. For instance, the nucleic acid molecules 502 may include ctDNA. The nucleic acid molecules 502, in various cases, are extracted from a sample, such as a biological sample obtained from a subject. In some implementations, the nucleic acid molecules 502 include DNA that is complementary to RNA present in the sample.

The nucleic acid molecules 502, in various cases, are ligated with adapters 504. For examples, the adapters 504 are hybridized to the nucleic acid molecules 502. The adapters 504, for example, include additional nucleic acid molecules. In various implementations, the adapters 504 have a shorter length than the nucleic acid molecules 502 being sequenced. For instance, the adapters 504 include amplification primers, flow cell adapter sequences, substrate adapter sequences, or sample index sequences. Although FIG. 5 illustrates adapters 504 being ligated to one end of each of the nucleic acid molecules 502, implementations are not so limited. For example, the adapters 504 may be ligated to both ends of each of the nucleic acid molecules 502.

In various examples, the nucleic acid molecules 502 ligated with the adapters 504 are amplified in order to generate amplified molecules 506. Various amplification techniques can be performed. For instance, the amplified molecules 506 are generated using PCR, a non-PCR amplification technique, an isothermal amplification technique, or any combination thereof.

Amplified molecules 506 may be captured by bait molecules 510 and sequenced. In some implementations, the amplified molecules 506 are sequenced via sequencing-by-synthesis. In various cases, fluorescently tagged deoxyribonucleotide triphosphates (dNTP) 512 are utilized to synthesize a strand that is complementary to DNA strands bound to the substrate 508. When a dNTP 512 is added to the strand (e.g., by an enzyme), the dNTP 512 emits an optical signal 514. In various implementations, the frequency of the optical signal 514 is dependent on the type of dNTP 512 from which the optical signal 514 is emitted. By detecting the optical signals 514 as the strand is being synthesized, the sequence of the original nucleic acid molecules 502 can be derived.

In some implementations, the amplified molecules 506 are sequenced via nanopore sequencing. For instance, the amplified molecules 506 are directed through a nanopore 516 extending through a substrate 518. In various cases, the amplified molecules 506 are negatively charged, such that they can be directed through the nanopore 516 by imposing an electrical field across the substrate 518. In various cases, the amplified molecules 506 and the nanopore 516 are in the presence of a charged solution. Thus, charged solutes traveling through the nanopore 516 can be monitored by reviewing an electrical signal (e.g., a current) sensed between electrodes 520 on either side of the substrate 518. As an amplified molecule 506 is directed through the nanopore 516, the individual bases within the amplified molecule 506 will block the nanopore 516, which may decrease the amount of charged solutes traveling through the nanopore 516 and consequently, the magnitude of the electrical signal detected by the electrodes 520. Each of the four types of bases within the amplified molecules 506, may block the nanopore 516 to a different extent. Therefore, the sequence of the nucleic acid molecules 502 can be derived by analyzing the measured electrical signal with respect to time as the amplified molecules 506 are directed through the nanopore 516.

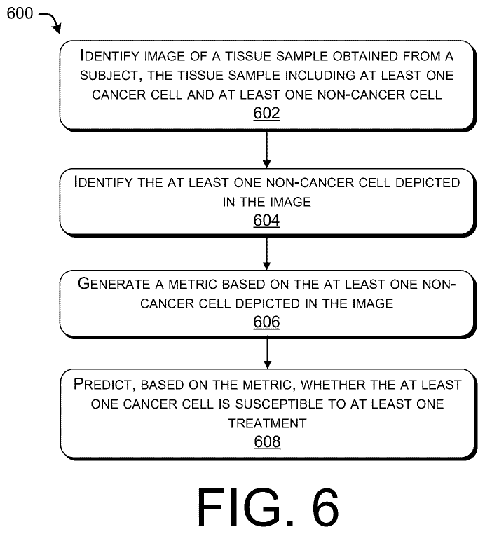

FIG. 6 illustrates an example process 600 for predicting cancer treatment efficacy based on characteristics of a tumor microenvironment. The process 600 can be performed by an entity, which may include at least one of a processor, computing device, medical device, a sequencer, an imaging device, an image analyzer, a predictive model, a genomic analyzer, a report generator, a clinical device, or any combination thereof.

At 602, the entity identifies an image of a tissue sample obtained from a subject. The tissue sample includes at least one cancer cell and at least one non-cancer cell. In various cases, the image depicts a section of the tissue sample that has been stained with a histological stain (e.g., H&E). For example, the image depicts the tissue sample after it has been fixed by a fixation agent. In various cases, the tissue sample is obtained from the subject via a tissue biopsy procedure. The non-cancer cell(s) may include one or more types of cells. For instance, the non-cancer cells may include at least one of an immune cell, a lymphocyte, a fibroblast, an endothelial cell, an epithelial cell, an erythrocyte, or a macrophage.

The image may be a 2D and/or a 3D image. In some cases, the image includes a 2D array of pixels. In some examples, the image includes a 3D array of voxels. In some cases, a 3D image is generated by stacking multiple 2D images representing multiple slices of the tissue sample obtained from the subject. Each pixel or voxel represents a region of a field-of-view (FOV) of the imaging device that has captured the image. For instance, an example pixel or voxel represents a region of a cell in the tissue sample. Each pixel or voxel of the image is defined by at least one value. For example, the image may be a color image including multiple color channels (e.g., a red channel, a green channel, and a blue channel in the case of an RGB image). An example pixel of a color image may have a first value corresponding to an amount of a first color in the region it represents (e.g., an amount of red in the region it represents), a second value corresponding to an amount of a second color in the region it represents (e.g., an amount of green in the region it represents), and a third value corresponding to an amount of a third color in the region it represents (e.g., an amount of blue in the region it represents).

In various cases, one or more imaging devices generate the image based on the tissue sample. For example, the imaging device(s) include an array of sensors (e.g., photosensors) configured to detect signals from the regions of the FOV of the imaging device(s). In some cases, the signals include light emitted by and/or reflected by portions of the tissue sample. According to some examples, the imaging device(s) include one or more light sources configured to illuminate the tissue sample in the FOV. In some cases, the entity includes the imaging device(s). In some examples, the entity receives data indicative of the image from the imaging device(s).

At 604, the entity identifies the at least one non-cancer cell depicted in the image. According to some examples, the entity performs image segmentation on the image. For example, the entity inputs the image into a computing model (e.g., an ML model, a CNN, a vision transformer, a vision kernel, or any combination thereof) configured to output a mask (e.g., a segmentation mask) indicating the at least one non-cancer cell depicted in the image. The mask, for instance, indicates at least one first pixel in the image depicting the non-cancer cells and at least one second pixel depicting one or more other structures. In some cases, the first pixel(s) have a first value and the second pixel(s) have a second value.

In some cases, the entity identifies the at least one non-cancer cell using a different type of image segmentation technique. In some cases, the entity performs an image recognition technique and identifies at least one nucleus of the at least one non-cancer cell depicted in the image. In some examples, the entity performs edge detection on the image to identify one or more boundaries of the at least one non-cancer cell.

At 606, the entity generates a metric based on the at least one non-cancer cell depicted in the image. The metric, in various cases, can represent a number, area, or density of the at least one non-cancer cell in the tissue sample. In some cases, the metric represents a number, area, or density of the at least one non-cancer cell in a portion (e.g., the stroma) of the tissue sample.

At 608, the entity predicts, based on the metric, whether the at least one cancer cell is susceptible to at least one treatment. In some examples, the entity compares the metric to a threshold. In some cases, the entity includes the metric in input data that is provided to a classifier, and the classifier outputs an indication of whether the at least one cancer cell is susceptible to the at least one treatment. The classifier, in some examples, includes one or more ML models that are pretrained to perform the classification. According to various cases, the treatment may include a chemotherapy, a radiotherapy, an immunotherapy, a targeted therapy, a surgical therapy, a cell-based therapy, or any combination thereof.

In some cases, the input data of the classifier includes additional types of data. For instance, the entity may include genomic features that are based on sequence read data of nucleic acid molecules obtained from the subject in the input data. In some cases, the entity includes an indication of cancer cell expression. For example, the entity may indicate whether the cancer cells express an antigen-of-interest associated with a particular immunotherapy in the input data.

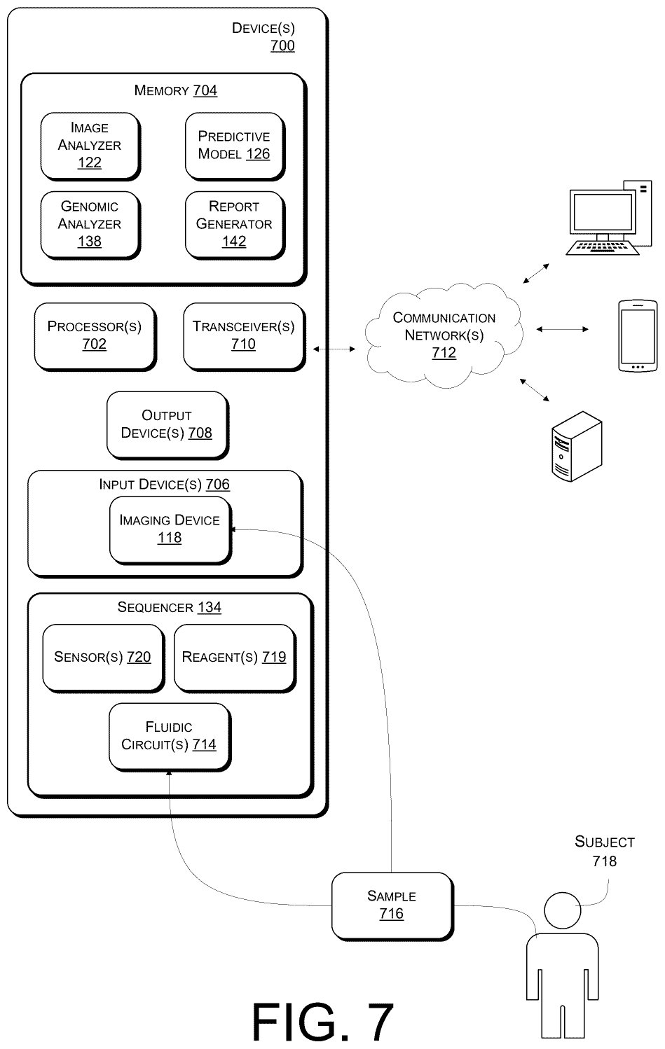

FIG. 7 illustrates one or more devices 700 configured to perform various operations described herein. The device(s) 700 include one or more processor(s) 702. In some implementations, the processor(s) 702 includes a central processing unit (CPU), a graphics processing unit (GPU), both CPU and GPU, or other processing unit or component known in the art.

The processor(s) 702 is operably connected to memory 704. In various implementations, the memory 704 is volatile (such as random access memory (RAM)), non-volatile (such as read only memory (ROM), flash memory, etc.) or some combination of the two. The memory 704 stores instructions that, when executed by the processor(s) 702, causes the processor(s) 702 to perform various operations. In various examples, the memory 704 stores methods, threads, processes, applications, objects, modules, any other sort of executable instruction, or a combination thereof. In some cases, the memory 704 stores files, databases, or a combination thereof. In some examples, the memory 704 includes, but is not limited to, RAM, ROM, electrically erasable programmable read-only memory (EEPROM), flash memory, or any other memory technology. In some examples, the memory 704 includes one or more of CD-ROMs, digital versatile discs (DVDs), content-addressable memory (CAM), or other optical storage, magnetic cassettes, magnetic tape, magnetic disk storage or other magnetic storage devices, or any other medium which can be used to store the desired information and which can be accessed by the processor(s) 702. For instance, the memory 704 stores instructions that, when executed by the processor(s) 702, causes the processor(s) 702 to perform operations of the image analyzer 122, the predictive model 126, the genomic analyzer 138, the report generator 142, or any combination thereof.

The processor(s) 702 is operably connected to one or more input devices 706 and one or more output devices 708. Collectively, the input device(s) 706 and the output device(s) 708 function as an interface between at least one user and the device(s) 700. The input device(s) 706 is configured to receive an input from a user and includes at least one of a keypad, a cursor control, a touch-sensitive display, a voice input device (e.g., a microphone), a haptic feedback device (e.g., a gyroscope), or any combination thereof. In some cases, the input device(s) 706 include the imaging device 118. The output device(s) 708 includes at least one of a display, a speaker, a haptic output device, a printer, or any combination thereof. In various examples, the processor(s) 702 causes a display among the input device(s) 706 to visually output various data described herein. In some implementations, the input device(s) 706 includes one or more touch sensors, the output device(s) 708 includes a display screen, and the touch sensor(s) are integrated with the display screen.

In various implementations, the processor(s) 702 is operably connected to one or more transceivers 710 that transmit and/or receive data over one or more communication networks 712. For example, the transceiver(s) 710 includes a network interface card (NIC), a network adapter, a local area network (LAN) adapter, or a physical, virtual, or logical address to connect to the various external devices and/or systems. In various examples, the transceiver(s) 710 includes any sort of wireless transceivers capable of engaging in wireless communication (e.g., radio frequency (RF) communication). For example, the communication network(s) 712 includes one or more wireless networks that include a 3rd Generation Partnership Project (3GPP) network, such as a Long Term Evolution (LTE) radio access network (RAN) (e.g., over one or more LTE bands), a New Radio (NR) RAN (e.g., over one or more NR bands), or a combination thereof. In some cases, the transceiver(s) 710 includes other wireless modems, such as a modem for engaging in WI-FI®, WIGIG®, WIMAX®, BLUETOOTH®, or infrared communication over the communication network(s) 712.

The device(s) 700 may further include the sequencer 134. In various implementations, the sequencer 134 includes one or more fluidic circuits 714 configured to receive a sample 716 derived from a subject 718. The sequencer 134, in various cases, may be configured to generate data indicative of one or more sequences of nucleic acid molecules (e.g., DNA and/or RNA) present in the sample 716. In various cases, the sequencer 134 introduces one or more reagents 719 to the fluidic circuit(s) 714 in order to prepare for and perform sequencing of the nucleic acid molecules. Further, the sequencer 134 may include one or more sensors 720 configured to measure or otherwise detect detection signals from the fluidic circuit(s) 714, which may be indicative of the sequences of the nucleic acid molecules. According to various implementations, the sensor(s) 720 may further include one or more ADCs. The sequencer 134, in various cases, outputs sequence read data to the processor(s) 702 for additional processing.

### EXPERIMENTAL EXAMPLE

The composition of the tumor microenvironment (TME) has been shown to influence response to immunotherapy (IO). In this study, digital pathology TME features of IO outcomes among patients with non-squamous, non-small cell lung cancer (NSCLC) within a real-world dataset were investigated.

### Methods

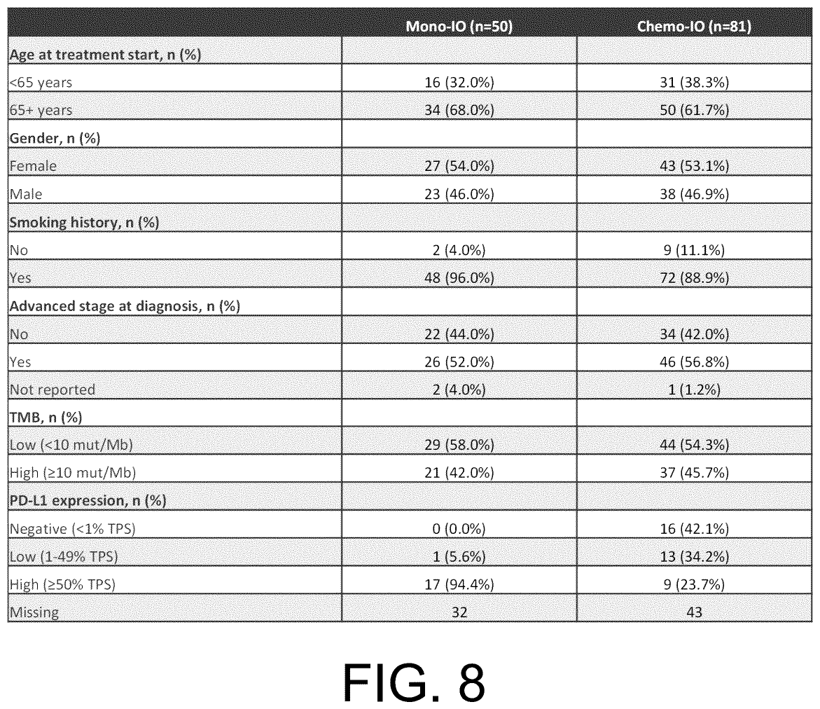

FIG. 8 summarizes the study population utilized in this Experimental Example. From the Flatiron Health-Foundation Medicine real-world clinico-genomic database, a cohort of 50 EGFR/ALK-negative, non-squamous NSCLC patients treated with first-line pembrolizumab monotherapy (mono-IO) or pembrolizumab in combination with carboplatin/cisplatin and pemetrexed (chemo-IO) were identified that had available whole slide images of H&E-stained tissue resection specimens. AI-powered tumor microenvironment (TME) characterization models developed by PathAI (Boston, MA; commercially available as PathExplore™) were deployed on scanned H&E slides to extract a panel of cell- and tissue-level human interpretable features (HIFs). Cox proportional hazards regression was used to identify digital pathology features or HIFs associated with overall survival.

### Results

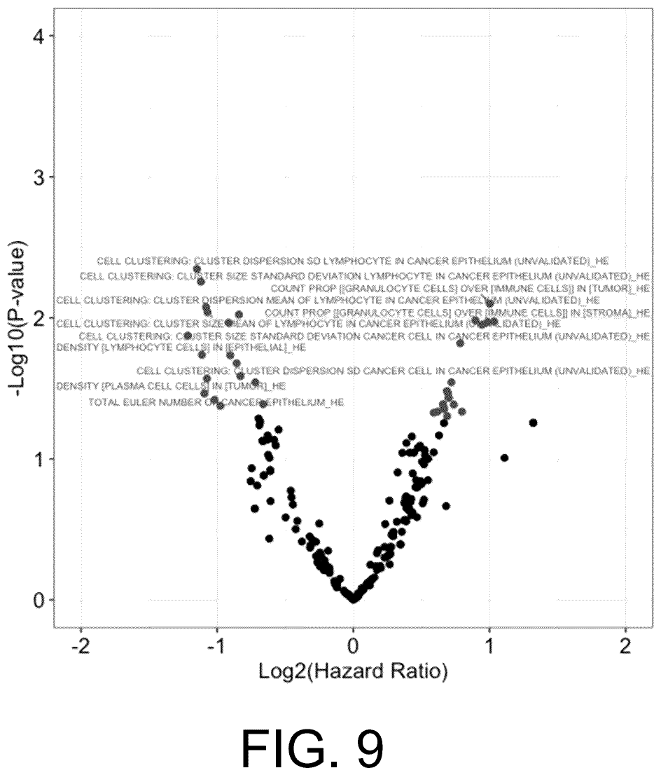

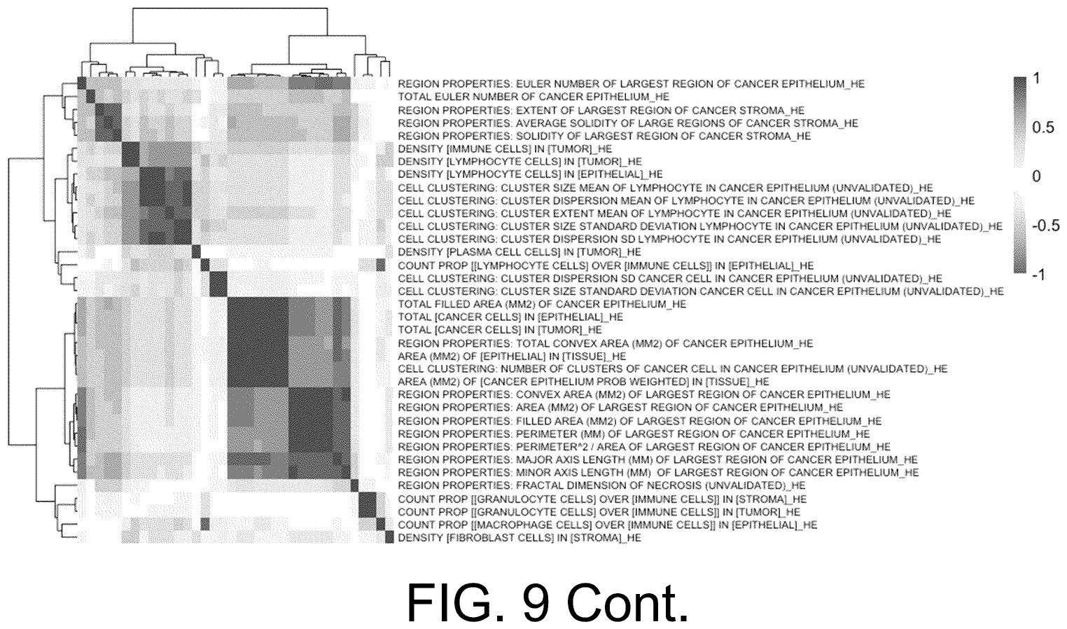

FIG. 9 illustrates a volcano plot representing HIFs associated with survival in mono-IO cohort and a heatmap of top HIFs observed in this example. As shown, different features associated with better or worse survival among patients that were treated with 1 L mono-immunotherapy. 17 HIFs were associated with better survival and 19 HIFs were associated with worse survival among patients treated with mono-IO (p<0.05).

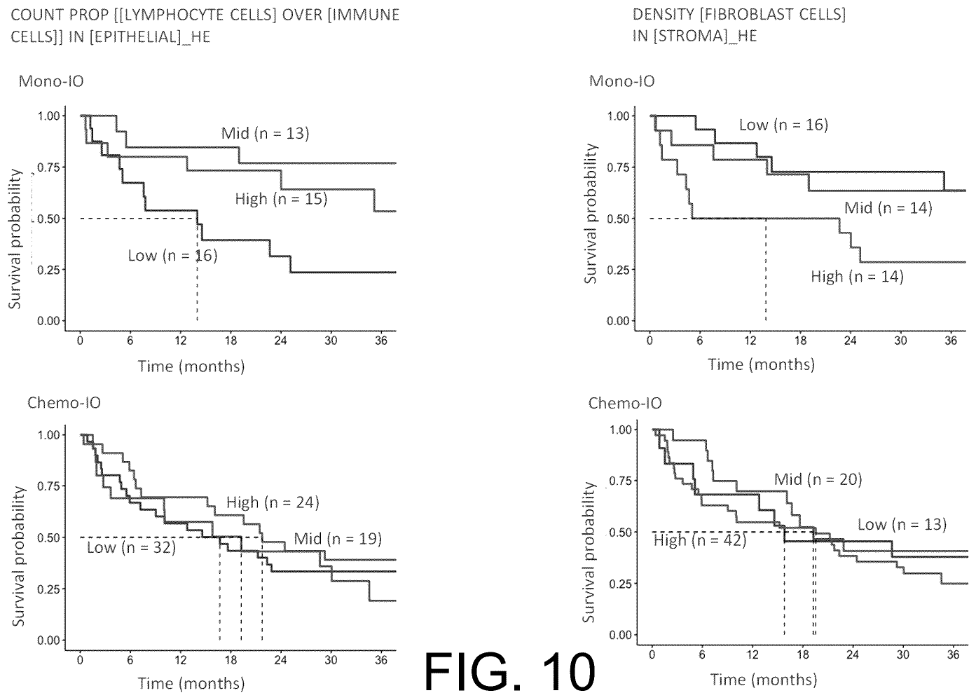

FIG. 10 illustrates Kaplan-Meier plots of overall survival for (top) mono-IO and (bottom) chemo-IO treated patients according to HIF status.

In this Example, among mono-IO treated patients, lymphocyte features were among the HIFs associated with favorable survival. An example of these features were ethe proportion of lymphocyte cells to total immune cells in the cancer epithelium (hazard ratio (HR) per unit standard deviation (SD)=0.61 [0.39, 0.95], p-value=0.03). Cell-level features associated with worse survival in the mono-IO cohort included density of fibroblast cells in stroma (HR per unit SD=1.51 [1.01, 2.25], p-value=0.05). The proportion of lymphocyte cells to total immune cells in cancer epithelium was associated with PD-L1 status (p<0.001). HIF-survival associations remained after adjusting for tumor mutational burden (TMB) and PD-L1 status. Additionally, similar associations were not observed among patients treated with chemo-IO.

High lymphocyte to immune cell proportion was associated with better survival compared to low lymphocyte to immune cell proportion in mono-IO treated patients ((adjusted HR per unit SD=0.32 [0.14, 0.73], p-value=0.01) but not in chemo-IO treated patients (adjusted HR per unit SD=1.00 [0.73, 1.38], p-value=1.0).

High fibroblast density was associated with worse survival compared to low fibroblast density in mono-IO treated patients (adjusted HR per unit SD=2.14 [1.23, 3.71], p-value=0.01) but not in chemo-IO treated patients (adjusted HR per unit SD=1.28 [0.89, 1.84], p-value=0.2).

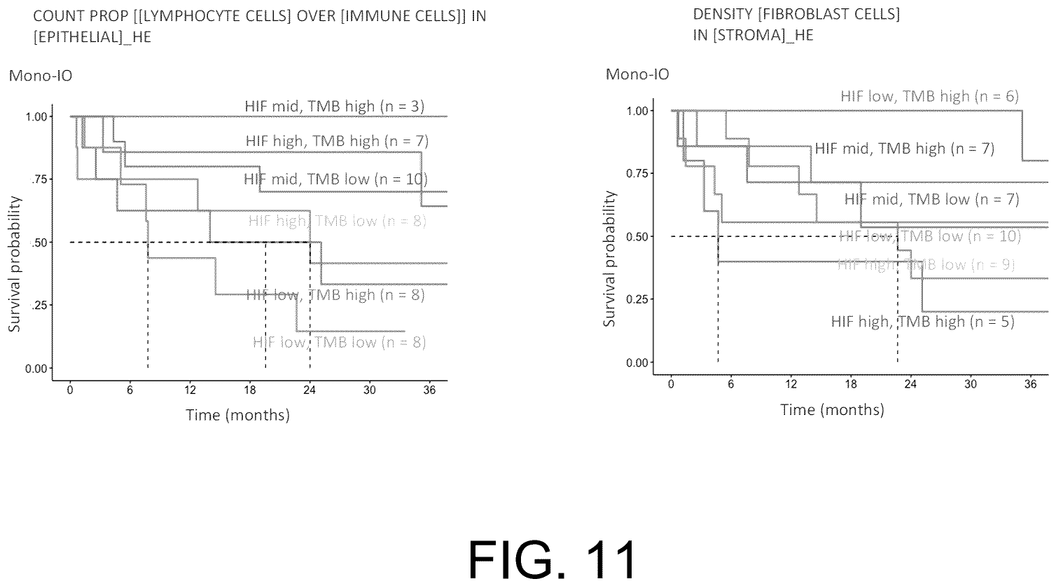

FIG. 11 illustrates Kaplan-Meier plots of overall survival for mono-IO treated patients stratified by HIF and TMB status. FIG. 11 illustrates that lymphocytic density and stromal fibroblast density can enhance the accuracy of effective treatment predictions based on TMB.

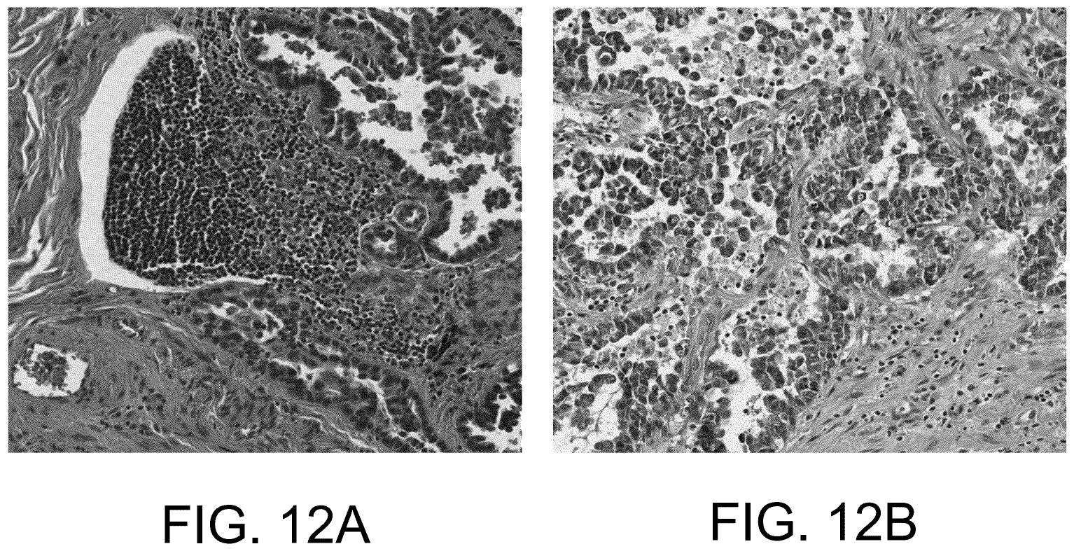

FIGS. 12A and 12B illustrate example tissue regions exhibiting strong lymphocyte presence (FIG. 12A) and tumor cells bordered by desmoplastic stroma with interspersed fibroblasts (FIG. 12B). FIG. 12A illustrates an example NSCLC (lung adenocarcinoma) patch with strong lymphocyte presence in and around epithelial tumor cells. FIG. 12B illustrates an example NSCLC (lung adenocarcinoma) patch with tumor cells and (in bottom right corner of patch) desmoplastic stroma with some presence of fibroblasts and lymphocytes.

These results indicate that the composition of TME assessed via digital pathology may have utility in identifying NSCLC patients that will respond to first-line immune checkpoint inhibitors beyond the established IO biomarkers. Addition of digital pathology evaluation of biopsy images to tumor profiling results may improve identification of patients in whom chemo-sparing IO regimens may be safe and effective.

### EXAMPLE CLAUSES

1. A method, including: capturing, by an imaging device, a first image of a tissue sample obtained from a subject that has been stained with hematoxylin and eosin (H&E), the tissue sample including at least one cancer cell, at least one lymphocyte, and at least one fibroblast; generating, using one or more processors, a segmentation mask by segmenting the at least one lymphocyte depicted in the first image; determining, using the one or more processors, a density of the at least one lymphocyte in the tissue sample based on the segmentation mask; determining, using the one or more processors, a density of the at least one fibroblast in stroma of the tissue sample by analyzing the first image; capturing, by the imaging device, a second image of the tissue sample that has been stained with an immunostain, the immunostain including an antibody that specifically binds a ligand; determining, using the one or more processors, whether the ligand is expressed by the at least one cancer cell by analyzing the second image; providing a plurality of nucleic acid molecules obtained from the subject; ligating one or more adapters onto one or more of the nucleic acid molecules from the plurality of nucleic acid molecules; capturing amplified nucleic acid molecules from the amplified nucleic acid molecules; sequencing, by a sequencer, all or a subset of the captured amplified nucleic acid molecules to obtain a plurality of sequence reads that represent the sequenced amplified nucleic acid molecules thereby generating sequence read data; receiving, at the one or more processors, the sequence read data for the plurality of sequence reads; determining, using the one or more processors, features of the subject based on the plurality of sequence reads; and predicting, using the one or more processors, whether the at least one cancer cell is susceptible to at least one treatment based on: the density of the at least one lymphocyte in the tissue sample; the density of the at least one fibroblast in the stroma of the tissue sample; whether the ligand is expressed by the at least one cancer cell; and the features of the subject.
2. The method of clause 1, wherein the imaging device includes a camera, and/or wherein the features of the subject include at least one of a mismatch repair deficiency (MMRD) probability score, a copy number state of at least one genetic locus, a fraction unstable score, a mutation signature, a tumor mutational burden (TMB) score, a presence of one or more hotspot mutations, a tumor purity, a presence of one or more aneuploidy events, or a presence of one or more pathogenic variants, and wherein the presence of one or more pathogenic variants are in one or more of polymerase E (POLE), TP53, CTNNNB1, L1CAM, PTEN, ERBB2, PMS2, MSH2, MSH6, MLH1, an estrogen receptor (ER) gene, or a progesterone receptor (PR) gene.
3. The method of clause 1 or 2, wherein determining, using the one or more processors, the density of the at least one lymphocyte in the tissue sample based on the segmentation mask includes: determining a number or area of the at least one lymphocyte in the tissue sample by analyzing the segmentation mask; determining a total area of the tissue sample depicted by the first image; and dividing the number or area of the at least one lymphocyte in the tissue sample by the total area of the tissue sample.
4. The method of any of clauses 1 to 3, wherein determining, using the one or more processors, the density of the at least one fibroblast in stroma of the tissue sample by analyzing the first image includes: identifying a boundary of the stroma in the tissue sample depicted in the first image; determining a number or area of the at least one fibroblast within the boundary of the stroma; determining an area of the stroma; and dividing the number or area of the at least one fibroblast within the boundary of the stroma.
5. The method of any of clauses 1 to 4, wherein the at least one treatment includes: a chemotherapy; an immunotherapy targeting the ligand and/or targeting a receptor for the ligand; or a combination of the chemotherapy or immunotherapy.
6. The method of any of clauses 1 to 5, wherein the ligand includes PD-L1, wherein the at least one treatment includes a PD-L1 inhibitor and/or a chemotherapy, and wherein the subject has non-squamous, non-small cell lung cancer (NSCLC).
7. A method, including: identifying an image of a tissue sample obtained from a subject, the tissue sample including at least one cancer cell and at least one non-cancer cell; identifying the at least one non-cancer cell depicted in the image; generating a metric based on the at least one non-cancer cell depicted in the image; and predicting, based on the metric, whether the at least one cancer cell is susceptible to at least one treatment.
8. The method of clause 7, wherein the image includes depicts a section of the tissue sample that has been stained with H&E.
9. The method of clause 7 or 8, wherein the image includes a section of the tissue sample that has been fixed by a fixation agent.
10. The method of any of clauses 7 to 9, wherein the tissue sample includes a tissue biopsy sample.
11. The method of any of clauses 7 to 10, wherein the at least one cancer cell includes at least one non-small cell lung cancer (NSCLC) cell.
12. The method of any of clauses 7 to 11, wherein the at least one non-cancer cell includes at least one immune cell.
13. The method of any of clauses 7 to 12, wherein the at least one non-cancer cell includes at least one lymphocyte.
14. The method of any of clauses 7 to 13, wherein the at least one non-cancer cell includes at least one of a fibroblast, an endothelial cell, an epithelial cell, a red blood cell, a macrophage, or an immune cell.
15. The method of any of clauses 7 to 14, wherein the image includes a two-dimensional (2D) image including a 2D array of pixels.
16. The method of any of clauses 7 to 15, wherein the image includes a three-dimensional (3D) image including a 3D array of voxels.
17. The method of clause 16, wherein identifying the image of the tissue sample obtained from the subject includes: identifying multiple 2D images of multiple slices of the tissue sample obtained from the subject; and generating the 3D image based on the multiple 2D images.
18. The method of any of clauses 7 to 17, wherein identifying the image of the tissue sample obtained from the subject includes: capturing, by a imaging device, the image.
19. The method of clause 18, wherein the imaging device includes a camera, and/or wherein identifying the image of the tissue sample obtained from the subject includes: receiving, from an external device, the image.
20. The method of any of clauses 7 to 19, wherein identifying the image of the tissue sample obtained from the subject includes: staining the tissue sample with at least one stain.
21. The method of any of clauses 7 to 20, wherein the subject is a human.
22. The method of any of clauses 7 to 21, wherein identifying the at least one non-cancer cell depicted in the image includes: performing image segmentation on the image.
23. The method of any of clauses 7 to 22, wherein identifying the at least one non-cancer cell depicted in the image includes: inputting the image into a computing model that is configured to output a mask indicating the at least one non-cancer cell depicted in the image.
24. The method of clause 23, wherein the computing model includes at least one of a convolutional neural network (CNN), a vision transformer, or a vision kernel,
25. The method of any of clauses 7 to 24, wherein identifying the at least one non-cancer cell depicted in the image includes: generating a segmentation mask indicating at least one first pixel in the image depicting the non-cancer cells and at least one second pixel depicting one or more other structures, and/or wherein identifying the at least one non-cancer cell depicted in the image includes identifying at least one nucleus of the at least one non-cancer cell depicted in the image.
26. The method of any of clauses 7 to 25, wherein identifying the at least one non-cancer cell depicted in the image includes identifying at least one boundary of the at least one non-cancer cell by performing edge detection on the image.
27. The method of any of clauses 7 to 26, wherein generating the metric based on the at least one non-cancer cell depicted in the image includes: determining at least one of a number, an area, or a density of the at least one non-cancer cell in the tissue sample.
28. The method of clause 27, wherein the at least one non-cancer cell includes at least one lymphocyte.
29. The method of any of clauses 7 to 28, wherein generating the metric based on the at least one non-cancer cell depicted in the image includes: determining at least one of a number, an area, or a density of the at least one non-cancer cell in stroma of the tissue sample.
30. The method of clause 29, wherein the at least one non-cancer cell includes at least one fibroblast.
31. The method of any of clauses 7 to 30, wherein predicting, based on the metric, whether the at least one cancer cell is susceptible to the at least one treatment includes: comparing the metric to a threshold.
32. The method of any of clauses 7 to 31, wherein predicting, based on the metric, whether the at least one cancer cell is susceptible to the at least one treatment includes: inputting the metric into a classifier configured to generate a classification of the at least one cancer cell, the classification indicating whether the at least one cancer cell is susceptible to the at least one treatment.
33. The method of any of clauses 7 to 32, wherein the at least one treatment includes a chemotherapy.
34. The method of any of clauses 7 to 33, wherein the at least one treatment includes an immunotherapy.
35. The method of any of clauses 7 to 34, wherein the at least one treatment includes at least one of chemotherapy, radiation therapy, immunotherapy, a targeted therapy, or surgery.
36. The method of any of clauses 7 to 35, further including: identifying sequence read data of nucleic acid molecules in the tissue sample or another sample obtained from the subject, wherein predicting, based on the metric, whether the at least one cancer cell is susceptible to the at least one treatment is further based on the sequence read data.
37. The method of clause 36, further including: receiving a plurality of nucleic acid molecules obtained from the tissue sample or an other sample obtained from the subject; ligating one or more adapters onto one or more nucleic acid molecules from the plurality of nucleic acid molecules; amplifying the one or more ligated nucleic acid molecules; capturing all or a subset of the amplified nucleic acid molecules; and sequencing, by a sequencer, the captured nucleic acid molecules to obtain a plurality of sequence reads that represent the captured nucleic acid molecules, thereby generating the sequence read data for a genome of the tissue sample or the other sample.
38. The method of clause 37, wherein the one or more adapters include amplification primers, flow cell adaptor sequences, substrate adapter sequences, or sample index sequences.
39. The method of clause 37 or 38, wherein the captured nucleic acid molecules are captured from the amplified nucleic acid molecules by hybridization to one or more bait molecules.
40. The method of clause 39, wherein the one or more bait molecules include one or more additional nucleic acid molecules, each of the one or more additional nucleic acid molecules including a region that is complementary to a region of a captured nucleic acid molecule.
41. The method of any of clauses 37 to 40, wherein amplifying the one or more ligated nucleic acid molecules includes performing a polymerase chain reaction (PCR) amplification technique, a non-PCR amplification technique, or an isothermal amplification technique.
42. The method of any of clauses 37 to 41, wherein sequencing the captured nucleic acid molecules includes use of a massively parallel sequencing (MPS) technique, whole genome sequencing (WGS), whole exome sequencing, targeted sequencing, direct sequencing, or Sanger sequencing.
43. The method of any of clauses 37 to 42, wherein sequencing the captured nucleic acid molecules includes next generation sequencing (NGS).
44. The method of any of clauses 37 to 43, wherein the sequencer includes a next generation sequencer.
45. The method of any of clauses 37 to 44, wherein sequencing the captured nucleic acid molecules includes sequencing-by-synthesis or nanopore sequencing.
46. The method of any of clauses 36 to 45, further including: generating ligated molecules by ligating adaptors onto nucleic acid molecules of the tissue sample or an other sample obtained from the subject;
generating amplified ligated molecules by amplifying the ligated molecules; generating, using the amplified ligated molecules, detection signals; detecting, by at least one sensor, the detection signals; and
generating the sequence read data based on the detection signals.
47. The method of clause 46, wherein the detection signals include electrical signals and/or optical signals.
48. The method of clause 46 or 47, wherein generating, using the amplified ligated molecules, the detection signals includes: synthesizing, by a polymerase using fluorescently tagged nucleotide triphosphates (NTPs), a synthesized nucleic acid molecule that is complementary to one of the amplified ligated molecules, and wherein detecting, by the at least one sensor, the detection signals includes: detecting, by at least one optical sensor, optical signals emitted by the fluorescently tagged NTPs upon binding to the synthesized nucleic acid molecule, the optical signals being indicative of at least one sequence of the nucleic acid molecules of the tissue sample or the other sample.
49. The method of any of clauses 46 to 48, wherein generating, using the amplified ligated molecules, the detection signals includes: directing the amplified ligated molecules through a nanopore extending from a first space to a second space through a substrate, and wherein detecting, by the at least one sensor, the detection signals includes: detecting, by sensors disposed in the first space and the second space, an electrical signal over time, the electrical signal being indicative of at least one sequence of the nucleic acid molecules of the tissue sample or the other sample.
50. The method of any of clauses 46 to 49, wherein the sequence read data indicates a full genome or RNA transcriptome of the tissue sample or the other sample.
51. The method of any of clauses 46 to 50, wherein the sequence read data indicates a whole exome of the tissue sample or the other sample.
52. The method of any of clauses 46 to 51, wherein the sequence read data indicates a predetermined panel of genes of the tissue sample or the other sample.
53. The method of clause 52, wherein the predetermined panel includes one or more of ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSFIR, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKC1, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, ZNF703, ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1B, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3Kδ, PIGF, PTCH, RAF, RANKL, RET, ROS1, SLAMF7, VEGF, VEGFA, or VEGFB.
54. The method of any of clauses 46 to 53, wherein the other sample includes a tissue biopsy sample or a liquid biopsy sample.
55. The method of clause 54, wherein the liquid biopsy sample and includes blood, plasma, cerebrospinal fluid, sputum, stool, urine, lymphatic fluid, or saliva.
56. The method of clause 54 or 55, wherein the liquid biopsy sample includes circulating cancer cells (CTCs).
57. The method of any of clauses 54 to 56, wherein the liquid biopsy sample includes cell-free DNA (cfDNA), circulating tumor DNA (ctDNA), or any combination thereof.
58. The method of any of clauses 46 to 57, further including extracting DNA or RNA from the tissue sample or the other sample.
59. The method of clause 58, wherein the DNA includes genomic DNA or cDNA.
60. The method of clause 58 or 59, wherein the RNA includes messenger RNA, microRNA, or non-coding RNA.
61. The method of any of clauses 46 to 60, further including: determining, based on the sequence read data, a mutational profile of the tissue sample or the other sample; inputting the mutational profile into a model, wherein the model is trained using training data related to a plurality of mutational signatures; and predicting one or more mutational signatures of the plurality of mutational signatures associated with the sample based on an output of the model, wherein the output of the model is associated with a dimensionality value that is less than a number of the plurality of mutational signatures, and wherein predicting whether the at least one cancer cell is susceptible to the at least one treatment is further based on the one or more mutational signatures.
62. The method of clause 61, wherein the model includes an autoencoder model.
63. The method of any of clauses 46 to 62, further including: determining, based on the sequence read data, a mismatch repair deficiency (MMRD) probability score, the MMRD probability score being indicative of a functional deficiency in at least one mismatch repair gene, wherein predicting whether the at least one cancer cell is susceptible to the at least one treatment is further based on the MMRD probability score.
64. The method of clause 63, wherein determining, based on the sequence read data, the MMRD probability score includes: generating, by extracting two or more features of the sequence read data, input data; and inputting the input data into a predictive model configured to generate the MMRD probability score based on the input data.
65. The method of any of clauses 46 to 64, further including: determining, based on the sequence read data, a copy number state, and wherein predicting whether the at least one cancer cell is susceptible to the at least one treatment is further based on the copy number state.
66. The method of clause 65, wherein determining, based on the sequence read data, the copy number state includes: generating, based on the sequence read data, a major allele coverage ratio and a minor allele coverage ratio; segmenting one or more nucleic acid sequences associated with the sequence read data into segments; generating copy number grid model input data including: a sum of the major allele coverage ratio and the minor allele coverage ratio; and a difference of the major allele coverage ratio and the minor allele coverage ratio; fitting copy number grid models including allowed copy number states to the copy number grid model input data; selecting a copy number grid model among the copy number grid models; and assigning the copy number state for at least a portion of the one or more nucleic acid sequences based on the selected copy number grid model.
67. The method of any of clauses 46 to 66, further including: determining, based on the sequence read data, a fraction unstable score, wherein predicting whether the at least one cancer cell is susceptible to the at least one treatment is further based on the fraction unstable score.
68. The method of clause 67, further including: determining an MSI fraction, the fraction unstable score including the MSI fraction.
69. The method of any of clauses 7 to 68, further including: obtaining a set of images of tissue samples obtained from a plurality of subjects, wherein: different subjects of the plurality of subjects have been treated via different immunotherapy treatments, and the tissue samples are indicative of tumor microenvironments associated with the plurality of subjects; obtaining survivorship information indicating survival times of the plurality of subjects; identifying, via a machine learning model, and based on the set of images, features of the tumor microenvironments that are predictive of the survival times.
70. The method of clause 69, wherein the features are identified via Cox proportional hazards regression.
71. The method of clause 69 or 70, wherein the different immunotherapy treatments include: monotherapy; and a combination of the monotherapy and chemotherapy.
72. The method of any of clauses 69 to 71, further including identifying particular features, of the features, that are associated with: individuals treated via a particular immunotherapy treatment, and survival times longer than a threshold period of time.
73. The method of clause 72, wherein: the at least one treatment is the particular immunotherapy treatment, the metric is a predicted survival time generated based on instances of the particular features indicated by the image, and predicting whether the at least one cancer cell is susceptible to the at least one treatment is based on the predicted survival time.
74. The method of any of clauses 7 to 73, the image being a first image depicting the tissue sample with a first stain, the method further including: identifying a second image of the tissue sample obtained from the subject, the second image depicting the tissue sample stained with a second stain, wherein predicting whether the at least one cancer cell is susceptible to the at least one treatment is further based on the second image.
75. The method of clause 74, wherein the second stain is an immunostain.
76. The method of clause 75, wherein the immunostain specifically binds a ligand expressed by one or more cancer cells.
77. The method of clause 76, wherein the at least one treatment includes an immunotherapy including an antibody that specifically binds to the ligand.
78. The method of any of clauses 7 to 77, further including: identifying morphological features of the at least one cancer cell depicted in the image, wherein predicting whether the at least one cancer cell is susceptible to the at least one treatment is further based on the morphological features of the at least one cancer cell.
79. The method of clause 78, wherein identifying the morphological features of the at least one cancer cell depicted in the image includes: identifying at least one boundary of the at least one cancer cell depicted in the image; and determining the morphological features based on the at least one boundary.
80. The method of clause 78 or 79, wherein identifying the morphological features of the at least one cancer cell depicted in the image includes: identifying at least one structure of the at least one cancer cell depicted in the image; and determining the morphological features based on the at least one structure.
81. The method of any of clauses 78 to 80, wherein the morphological features include at least one of a nucleus size, a variance of cell size, a variance of cell shape, a cytoplasm color, or a nucleoli prominence.
82. The method of any of clauses 7 to 81, wherein predicting, based on the metric, whether the at least one cancer cell is susceptible to at least one treatment includes: predicting that the at least one cancer cell is susceptible to a first treatment.
83. The method of clause 82, further including: outputting a recommendation to administer the first treatment.
84. The method of clause 82 or 83, further including: administering the first treatment to the subject.
85. The method of any of clauses 7 to 84, wherein predicting, based on the metric, whether the at least one cancer cell is susceptible to at least one treatment includes: predicting that the at least one cancer cell is resistant to a second treatment.
86. The method of clause 85, further including: outputting a recommendation to refrain from administering the second treatment.
87. The method of any of clauses 7 to 86, further including: generating, based on predicting whether the at least one cancer cell is susceptible to the at least one treatment, a genomic profile of the subject.
88. The method of clause 87, wherein the genomic profile includes results from at least one of: a comprehensive genomic profiling test; a gene expression profiling test; a cancer hotspot panel test; a DNA methylation test; a DNA fragmentation test; or an RNA fragmentation test.
89. The method of clause 88, wherein the genomic profile of the subject includes results from a nucleic acid sequencing-based test.
90. The method of clause 88 or 89, further including: selecting, based on the genomic profile, an anticancer agent for administration to the subject.
91. The method of clause 90, further including: administering the anticancer agent to the subject.
92. The method of any of clauses 7 to 91, further including: generating a report indicating whether the at least one cancer cell is susceptible to the at least one treatment; and outputting the report.
93. The method of clause 92, wherein outputting the report includes: transmitting data indicating the report to an external device.
94. The method of clause 93, wherein the external device is associated with the subject and/or a healthcare provider.
95. The method of clause 93 or 94, wherein the data is transmitted over one or more communication networks.
96. The method of any of clauses 93 to 95, wherein the data is transmitted over a peer-to-peer connection.
97. The method of any of clauses 93 to 96, wherein outputting the report includes: visually presenting, by a display, the report.
98. The method of any of clauses 7 to 97, further including: determining, based on predicting whether the at least one cancer cell is susceptible to the at least one treatment, whether the subject is eligible for a clinical trial.
99. The method of any of clauses 7 to 98, wherein the method is executed by one or more processors.
100. A system comprising at least one processor configured to perform the method of any of claims 1 to 99.
101. A non-transitory computer-readable medium configured to perform the method of any of claims 1 to 99.
102. A system, including: at least one processor; and memory storing instructions that, when executed by the at least one processor, cause the at least one processor to perform operations including: identifying an image of a tissue sample obtained from a subject, the tissue sample including at least one cancer cell and at least one non-cancer cell; identifying the at least one non-cancer cell depicted in the image; generating a metric based on the at least one non-cancer cell depicted in the image; and predicting, based on the metric, whether the at least one cancer cell is susceptible to at least one treatment.
103. The system of clause 102, further including: a imaging device configured to capture the image of the tissue sample.
104. The system of clause 102 or 103, further including: a transceiver configured to transmit data indicating whether the at least one cancer cell is predicted to be susceptible to the at least one treatment.
105. The system of any of clauses 102 to 104, further including: an output device configured to indicate whether the at least one cancer cell is predicted to be susceptible to the at least one treatment.
106. The system of any of clauses 102 to 105, further including: a sequencer configured to generate sequence read data based on nucleic acid molecules obtained from the tissue sample or another sample obtained from the subject, wherein predicting whether the at least one cancer cell is susceptible to the at least one treatment is further based on the sequence read data.
107. A method of identifying an individual having a cancer who may benefit from a treatment comprising an immunotherapy, the method comprising detecting in a tissue sample from the individual at least one tumor microenvironment (TME) feature comprising at least one of: a density of at least one fibroblast cell in stroma of the tissue sample; a proportion of at least one macrophage cell to immune cells in epithelium of the tissue sample; a proportion of at least one granulocyte cell to immune cells in tumor of the tissue sample; a proportion of at least one granulocyte cell to immune cells in the stroma of the tissue sample; a minor axis length of cancer epithelium in the tissue sample; a major axis length of the cancer epithelium in the tissue sample; a perimeter of the cancer epithelium in the tissue sample; an area of the cancer epithelium in the tissue sample; a square of the perimeter of the cancer epithelium in the tissue sample; a convex area of the cancer epithelium in the tissue sample; an area of epithelium in the tissue sample; a number of cancer cells in the tissue sample; a number of cancer cells in the epithelium in the tissue sample; a total filled area of the cancer epithelium; a proportion of at least one lymphocyte to the immune cells in the epithelium of the tissue sample; a density of plasma cells in the tumor of the tissue sample; a density of at least one lymphocyte in the epithelium of the tissue sample; a density of at least one lymphocyte in the tumor of the tissue sample; a density of at least one immune cell in the tumor of the tissue sample; a solidity of cancer stroma in the tissue sample; an extent of the cancer stroma in the tissue sample; an Euler number of the cancer epithelium in the tissue sample; a mean cluster size of the at least one lymphocyte in the cancer epithelium; a mean cluster dispersion of the at least one lymphocyte in the cancer epithelium; a mean cluster extent of lymphocytes in the cancer epithelium; a standard deviation of the cluster size of the at least one lymphocyte in the cancer epithelium; a standard deviation of the cluster dispersion of the at least one lymphocyte in the cancer epithelium; a standard deviation of a cluster dispersion of cancer cells in the cancer epithelium; a standard deviation of a cluster size of the cancer cells in the cancer epithelium; or a number of clusters of the cancer cells in the cancer epithelium, wherein detection of the at least one TME feature in the tissue sample identifies the individual as one who may benefit from the immunotherapy.
108. A method of treating or delaying progression of a cancer in an individual in need thereof, comprising: acquiring knowledge of at least one of: a density of at least one fibroblast cell in stroma of the tissue sample; a proportion of at least one macrophage cell to immune cells in epithelium of the tissue sample; a proportion of at least one granulocyte cell to immune cells in tumor of the tissue sample; a proportion of at least one granulocyte cell to immune cells in the stroma of the tissue sample; a minor axis length of cancer epithelium in the tissue sample; a major axis length of the cancer epithelium in the tissue sample; a perimeter of the cancer epithelium in the tissue sample; an area of the cancer epithelium in the tissue sample; a square of the perimeter of the cancer epithelium in the tissue sample; a convex area of the cancer epithelium in the tissue sample; an area of epithelium in the tissue sample; a number of cancer cells in the tissue sample; a number of cancer cells in the epithelium in the tissue sample; a total filled area of the cancer epithelium; a proportion of at least one lymphocyte to the immune cells in the epithelium of the tissue sample; a density of plasma cells in the tumor of the tissue sample; a density of at least one lymphocyte in the epithelium of the tissue sample; a density of at least one lymphocyte in the tumor of the tissue sample; a density of at least one immune cell in the tumor of the tissue sample; a solidity of cancer stroma in the tissue sample; an extent of the cancer stroma in the tissue sample; an Euler number of the cancer epithelium in the tissue sample; a mean cluster size of the at least one lymphocyte in the cancer epithelium; a mean cluster dispersion of the at least one lymphocyte in the cancer epithelium; a mean cluster extent of lymphocytes in the cancer epithelium; a standard deviation of the cluster size of the at least one lymphocyte in the cancer epithelium; a standard deviation of the cluster dispersion of the at least one lymphocyte in the cancer epithelium; a standard deviation of a cluster dispersion of cancer cells in the cancer epithelium; a standard deviation of a cluster size of the cancer cells in the cancer epithelium; or a number of clusters of the cancer cells in the cancer epithelium, responsive to said knowledge, administering to the individual an effective amount of a treatment that comprises an immunotherapy.
109. A method of treating or delaying progression of a cancer in an individual in need thereof, including: acquiring knowledge of at least one of: a density of at least one fibroblast cell in stroma of a tissue sample from the individual, or a density of at least one lymphocyte in the tissue sample; and responsive to said knowledge, administering to the individual an effective amount of a treatment that includes an immunotherapy.
110. The method of clause 109, wherein acquiring said knowledge further includes acquiring knowledge of an RNA transcriptome of the tissue sample.
111. The method of clause 109 or 110, wherein the treatment includes: a monotherapy including the immunotherapy; or a combination of the monotherapy and a chemotherapy.
112. The method of any of clauses 109 to 111, wherein the immunotherapy includes a PD-L1 inhibitor, and wherein the individual has non-squamous, non-small cell lung cancer (NSCLC).

### CONCLUSION

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be used for realizing implementations of the disclosure in diverse forms thereof.

As will be understood by one of ordinary skill in the art, each implementation disclosed herein can comprise, consist essentially of or consist of its particular stated element, step, or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the implementation to the specified elements, steps, ingredients or components and to those that do not materially affect the implementation. As used herein, the term “based on” is equivalent to “based at least partly on,” unless otherwise specified.

Unless otherwise indicated, all numbers expressing quantities, properties, conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e., denoting somewhat more or somewhat less than the stated value or range, to within a range of +20% of the stated value; +19% of the stated value; +18% of the stated value; +17% of the stated value; +16% of the stated value; +15% of the stated value; +14% of the stated value; +13% of the stated value; +12% of the stated value; +11% of the stated value; +10% of the stated value; +9% of the stated value; +8% of the stated value; +7% of the stated value; +6% of the stated value; +5% of the stated value; +4% of the stated value; +3% of the stated value; +2% of the stated value; or +1% of the stated value.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the,” and similar referents used in the context of describing implementations (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate implementations of the disclosure and does not pose a limitation on the scope of the disclosure. No language in the specification should be construed as indicating any non-claimed element essential to the practice of implementations of the disclosure.

Groupings of alternative elements or implementations disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, the practice of the present disclosure can employ conventional techniques of immunology, molecular biology, microbiology, cell biology and recombinant DNA. These methods are described in the following publications. See, e.g., Sambrook, et al. Molecular Cloning: A Laboratory Manual, 2nd Edition (1989); F. M. Ausubel, et al. eds., Current Protocols in Molecular Biology, (1987); the series Methods IN Enzymology (Academic Press, Inc.); M. MacPherson, et al., PCR: A Practical Approach, IRL Press at Oxford University Press (1991); MacPherson et al., eds. PCR 2: Practical Approach, (1995); Harlow and Lane, eds. Antibodies, A Laboratory Manual, (1988); and R. I. Freshney, ed. Animal Cell Culture (1987).

Tumor mutational burden (TMB) is a measure of the number of mutations carried by tumor cells. By comparing DNA sequences from a patient's healthy tissues and tumor cells, the number of acquired somatic mutations present in tumors, but not in normal tissues, may be determined. In some instances, driver mutations may be excluded from a TMB calculation.

In certain examples, “tumor mutational burden” or “TMB” refers to the number of somatic mutations in a tumor's genome and/or the number of somatic mutations per area of the tumor's genome. In some embodiments, TMB, as used herein, refers to the number of somatic mutations per megabase (Mb) of DNA sequenced. In some embodiments, germline (inherited) variants are excluded when determining TMB, given that the immune system has a higher likelihood of recognizing these as self. In various cases, driver mutations are excluded from a TMB calculation.

Microsatellites are highly polymorphic DNA-repeat regions. In certain examples, “microsatellite” refers to a repetitive nucleic acid having repeat units of less than about 10 base pairs or nucleotides in length. In certain examples, a microsatellite refers to a tract of tandemly repeated (i.e. adjacent) DNA motifs ranging from one to six or up to ten nucleotides, with each motif repeated 5 to 50 repeated times. “Microsatellite instability” refers to genetic instability in the microsatellite regions. Cancer patients with microsatellite instability classified as being bigh (MSI-H or MSI-High) frequently exhibit an accumulation of somatic mutations in tumor cells that leads to a range of molecular and biological changes including high tumor mutational burden, increased expression of neoantigens and abundant tumor-infiltrating lymphocytes. Chang et al. “Microsatellite Instability: A Predictive Biomarker for Cancer Immunotherapy,” Appl Immunohistochem Mol Morphol, 26(2):e15-e21 (2018). These changes have been linked to increased sensitivity to checkpoint inhibitor drugs, such as pembrolizumab, which is used to treat advanced melanoma, head and neck squamous cell carcinoma, non-small cell lung cancer (NSCLC), and classical Hodgkin lymphoma.

A viral status test refers to a test that identifies the presence of viral RNA or DNA in a subject. The test can identify viral load and/or viral identity. For example, the viral status test can identify the presence of viral RNA or DNA associated with the occurrence of certain cancers. Examples of such viruses include Hepatitis B Virus (HBV) and Hepatitis C Virus (HCV), Kaposi Sarcoma-Associated Herpesvirus (KSHV), Merkel Cell Polyomavirus (MCV), Human Papillomavirus (HPV), Human Immunodeficiency Virus Type 1 (HIV-1, or HIV), Human T-Cell Lymphotropic Virus Type 1 (HTLV-1), and Epstein-Barr Virus (EBV). Cancer “hotspot” mutations give rise to oncological outcomes. PhyloP, SIFT, Grantham, COSMIC and PolyPhen-2 are in silico tools that can be used to assess pathogenicity of identified variants. Exemplary hotspot genes and mutations include EGFR exon 19 activating mutation, EGFR exon 19 deletion, EGFR exon 19 insertion, EGFR exon 19 sensitizing mutation, EGFR exon 20 activation mutation, EGFR exon 20 insertion, EGFR G719 mutation, EGFR L858R mutation, EGFR L861 mutation, EGFR S768 mutation, EGFR T790M mutation, C797 mutation, KIT activating mutation, KRAS activating mutation, MET activating mutation, NRAS activating mutation, PMS2 promoter mutations, among many others. Hotspot mutations also occur in the following genes: AKT2, BRCA1, BRCA2, ERC1, NSD1, POLH, PPM1G, PTEN, RAD18, RAD51, RAD51B, RB1, TERT, TP53, TP53Bp1, ALK, ARMT1, ATAD5, ATG7, ATIC, AXL, BIRC6, BRD3, BRD4, CAPRIN1, CCAR2, CCDC6, CDK5RAP2, CHD9, CIT, CTNNB1, CUL1, EBF1, EIF3E, HIP1, HMGA2, IRF2BP2, NOTCH1, NOTCH4, NPM1, OFD1, TACC1, TACC3, TERF2, TMEM106B, UBE2L3, USP10, WRDR48, YAP1, ZEB2, and ZMYND8.

A “DNA methylation test” refers to an assay, which can be commercially available, for distinguishing methylated versus unmethylated cytosine loci in DNA. Techniques for measuring cytosine methylation include bisulfite-based methylation assays. The addition of bisulfite to DNA results in the methylation of unmethylated cytosine and its ultimate conversion to the nucleotide uracil. Uracil has similar binding properties to thiamine in the DNA sequence. Previously methylated cytosine does not undergo similar chemical conversion on exposure to bisulfite. Bisulfite assays can thus be used to discriminate previously methylated versus unmethylated cytosine.

An exemplary quantitative methylation detection assay combines bisulfite treatment and restriction analysis COBRA, which uses methylation sensitive restriction endonucleases, gel electrophoresis, and detection based on labeled hybridization probes. (Ziong and Laird, Nucleic Acid Res. 1997 25; 2532-4). Another exemplary detection assay is the methylation specific polymerase chain reaction PCR (MSPCR) for amplification of DNA segments of interest. This assay can be performed after sodium bisulfite conversion of cytosine and uses methylation sensitive probes. Other detection assays include the Quantitative Methylation (QM) assay, which combines PCR amplification with fluorescent probes designed to bind to putative methylation sites; MethyLight™ (Qiagen, Redwood City, CA) a quantitative methylation detection assay that uses fluorescence-based PCR (Eads, et al., Cancer Res. 1999; 59:2302-2306); and Ms-SNuPE, a quantitative technique for determining differences in methylation levels in CpG sites. As with other techniques, Ms-SNuPE also requires bisulfite treatment to be performed first, leading to the conversion of unmethylated cytosine to uracil while methyl cytosine is unaffected. PCR primers specific for bisulfite converted DNA are then used to amplify the target sequence of interest. The amplified PCR product is isolated and used to quantitate the methylation status of the CpG site of interest. (Gonzalgo and Jones Nuclei Acids Res1997; 25:252-31).

In particular embodiments, pyrosequencing can be used to detect marker methylation. Pyrosequencing is a method of DNA sequencing that relies on detection of the release of pyrophosphates as DNA is synthesized (and is therefore a “sequencing by synthesis” technique). To assess methylation by pyrosequencing, a DNA sample can be incubated with sodium bisulfite, converting unmethylated cytosine to uracil. The presence of uracil will result in thymine incorporation during PCR amplification. Therefore, sequencing results that include thymine at a nucleotide position that is known to encode cytosine can be interpreted as unmethylated sites. In contrast cytosines present in the sequencing results indicate that the site was methylated in the original DNA sample, because methylation protects cytosine from conversion to uracil upon treatment. Bisulfite treatment can also be performed on control samples with known methylation patterns, to reduce or eliminate false positive results. Commercially available pyrosequencing machines include Pyro Mark Q96 (Qiagen, Hilden, Germany). For more details on methods to use pyrosequencing for measurement of methylation, see Delaney et al. Methods Mol Biol. 2015 1343:249-264. Pyrosequencing is especially useful for detecting methylation in the CpG sites within genes.

In particular embodiments, a protein marker is detected by contacting a sample with reagents (e.g., antibodies), generating complexes of reagent and marker(s), and detecting the complexes. Particular embodiments for detecting and measuring protein levels can use methods including agglutination, chemiluminescence, electro-chemiluminescence (ECL), enzyme-linked immunoassays (ELISA), immunoassay, immunoblotting, immunodiffusion, immunoelectrophoresis, immunofluorescence, immunohistochemistry, immunoprecipitation, mass-spectrometry, and western blot. See also, e.g., E. Maggio, Enzyme-Immunoassay (1980), CRC Press, Inc., Boca Raton, Fla; and U.S. Pat. Nos. 4,727,022; 4,659,678; 4,376,110; 4,275,149; 4,233,402; and 4,230,797.

Read depth refers to the number of times that a specific genomic site is sequenced during a sequencing run.

Certain implementations are described herein, including the best mode known to the inventors for carrying out implementations of the disclosure. Of course, variations on these described implementations will become apparent to those of ordinary skill in the art upon reading the foregoing description. The inventor expects skilled artisans to employ such variations as appropriate, and the inventors intend for implementations to be practiced otherwise than specifically described herein. Accordingly, the scope of this disclosure includes all modifications and equivalents of the subject matter recited in the claims appended hereto as permitted by applicable law. Moreover, any combination of the above-described elements in all possible variations thereof is encompassed by implementations of the disclosure unless otherwise indicated herein or otherwise clearly contradicted by context.

What is claimed is:

1. A method, comprising:
capturing, by an imaging device, a first image of a tissue sample obtained from a subject that has been stained with hematoxylin and eosin (H&E), the tissue sample comprising at least one cancer cell, at least one lymphocyte, and at least one fibroblast;
generating, using one or more processors, a segmentation mask by segmenting the at least one lymphocyte depicted in the first image;
determining, using the one or more processors, a density of the at least one lymphocyte in the tissue sample based on the segmentation mask;
determining, using the one or more processors, a density of the at least one fibroblast in stroma of the tissue sample by analyzing the first image;
capturing, by the imaging device, a second image of the tissue sample that has been stained with an immunostain, the immunostain comprising an antibody that specifically binds a ligand;
determining, using the one or more processors, whether the ligand is expressed by the at least one cancer cell by analyzing the second image;
providing a plurality of nucleic acid molecules obtained from the subject;
ligating one or more adapters onto one or more nucleic acid molecules from the plurality of nucleic acid molecules;
capturing amplified nucleic acid molecules from the amplified nucleic acid molecules;
sequencing, by a sequencer, all or a subset of the captured amplified nucleic acid molecules to obtain a plurality of sequence reads that represent the sequenced amplified nucleic acid molecules thereby generating sequence read data;
receiving, at the one or more processors, the sequence read data for the plurality of sequence reads;
determining, using the one or more processors, features of the subject based on the plurality of sequence reads; and
predicting, using the one or more processors, whether the at least one cancer cell is susceptible to at least one treatment based on:
the density of the at least one lymphocyte in the tissue sample;
the density of the at least one fibroblast in the stroma of the tissue sample;
whether the ligand is expressed by the at least one cancer cell; and
the features of the subject.

2. The method of claim 1, wherein the imaging device comprises a camera.

3. The method of claim 1, wherein the features of the subject comprise at least one of a mismatch repair deficiency (MMRD) probability score, a copy number state of at least one genetic locus, a fraction unstable score, a mutation signature, a tumor mutational burden (TMB) score, a presence of one or more hotspot mutations, a tumor purity, a presence of one or more aneuploidy events, or a presence of one or more pathogenic variants, and
wherein the presence of one or more pathogenic variants are in one or more of polymerase E (POLE), TP53, CTNNNB1, L1CAM, PTEN, ERBB2, PMS2, MSH2, MSH6, MLH1, an estrogen receptor (ER) gene, or a progesterone receptor (PR) gene.

4. The method of claim 1, wherein determining, using the one or more processors, the density of the at least one lymphocyte in the tissue sample based on the segmentation mask comprises:
determining a number or area of the at least one lymphocyte in the tissue sample by analyzing the segmentation mask;
determining a total area of the tissue sample depicted by the first image; and
dividing the number or area of the at least one lymphocyte in the tissue sample by the total area of the tissue sample, and
wherein determining, using the one or more processors, the density of the at least one fibroblast in stroma of the tissue sample by analyzing the first image comprises:
identifying a boundary of the stroma in the tissue sample depicted in the first image;
determining a number or area of the at least one fibroblast within the boundary of the stroma;
determining an area of the stroma; and
dividing the number or area of the at least one fibroblast within the boundary of the stroma.

5. A method of treating or delaying progression of a cancer in an individual in need thereof, comprising:
acquiring knowledge of at least one of:
a density of at least one fibroblast cell in stroma of a tissue sample from the individual, or
a density of at least one lymphocyte in the tissue sample; and
responsive to said knowledge, administering to the individual an effective amount of a treatment that comprises an immunotherapy.

6. The method of claim 5, wherein acquiring said knowledge further comprises acquiring knowledge of an RNA transcriptome of the tissue sample.

7. The method of claim 5, wherein the treatment comprises:
a monotherapy comprising the immunotherapy; or
a combination of the monotherapy and a chemotherapy.

8. The method of claim 5, wherein the immunotherapy comprises a PD-L1 inhibitor, and
wherein the individual has non-squamous, non-small cell lung cancer (NSCLC).

9. A method, comprising:
identifying an image of a tissue sample obtained from a subject, the tissue sample comprising at least one cancer cell and at least one non-cancer cell;
identifying the at least one non-cancer cell depicted in the image;
generating a metric based on the at least one non-cancer cell depicted in the image; and
predicting, based on the metric, whether the at least one cancer cell is susceptible to at least one treatment.

10. The method of claim 9, wherein the at least one non-cancer cell comprises at least one of a lymphocyte, a fibroblast, an endothelial cell, an epithelial cell, a red blood cell, a macrophage, or an immune cell.

11. The method of claim 9, wherein identifying the image of the tissue sample obtained from the subject comprises:
capturing, by an imaging device, the image.

12. The method of claim 9, wherein identifying the at least one non-cancer cell depicted in the image comprises:
inputting the image into a computing model that is configured to output a mask indicating the at least one non-cancer cell depicted in the image, and
wherein the computing model comprises at least one of a convolutional neural network (CNN), a vision transformer, or a vision kernel.

13. The method of claim 9, wherein identifying the at least one non-cancer cell depicted in the image comprises:
identifying at least one nucleus of the at least one non-cancer cell depicted in the image; and/or
identifying at least one boundary of the at least one non-cancer cell by performing edge detection on the image.

14. The method of claim 9, wherein generating the metric based on the at least one non-cancer cell depicted in the image comprises:
determining at least one of a number, an area, or a density of the at least one non-cancer cell in the tissue sample.

15. The method of claim 9, wherein generating the metric based on the at least one non-cancer cell depicted in the image comprises:
determining at least one of a number, an area, or a density of the at least one non-cancer cell in stroma of the tissue sample,
wherein the at least one non-cancer cell comprises at least one fibroblast.

16. The method of claim 9, wherein predicting, based on the metric, whether the at least one cancer cell is susceptible to the at least one treatment comprises:
comparing the metric to a threshold; or
inputting the metric into a classifier configured to generate a classification of the at least one cancer cell, the classification indicating whether the at least one cancer cell is susceptible to the at least one treatment, and
wherein the at least one treatment comprises at least one of chemotherapy, radiation therapy, immunotherapy, a targeted therapy, or surgery.

17. The method of claim 9, further comprising:
obtaining a set of images of tissue samples obtained from a plurality of subjects, wherein:
different subjects of the plurality of subjects have been treated via different immunotherapy treatments, and
the tissue samples are indicative of tumor microenvironments associated with the plurality of subjects;
obtaining survivorship information indicating survival times of the plurality of subjects; and
identifying, via a machine learning model, and based on the set of images, features of the tumor microenvironments that are predictive of the survival times.

18. The method of claim 17, wherein the different immunotherapy treatments comprise:
monotherapy; and
a combination of the monotherapy and chemotherapy.

19. The method of claim 9, the image being a first image depicting the tissue sample with a first stain, the method further comprising:
identifying a second image of the tissue sample obtained from the subject, the second image depicting the tissue sample stained with a second stain, the second stain comprising an immunostain that specifically binds to a ligand,
wherein predicting whether the at least one cancer cell is susceptible to the at least one treatment is further based on the second image, and
wherein the at least one treatment comprises an immunotherapy comprising an antibody that specifically binds to the ligand.

20. The method of claim 9, further comprising:
generating a report indicating whether the at least one cancer cell is susceptible to the at least one treatment; and
outputting the report.
